(19) United States
(12) Patent Application Publication
(10) Pub. No.: US 2010/0099762 A1
(43) Pub. Date: Apr. 22, 2010

(54) COMBINATION THERAPY

(75) Inventors: Ashley I. Bush, Parkville (AU); David L. Copolov, East Malvern (AU); Michael Berk, Highton (AU)

Correspondence Address: SEED INTELLECTUAL PROPERTY LAW GROUP PLLC, 701 FIFTH AVE, SUITE 5400, SEATTLE, WA, 98104, US

(73) Assignee: THE MENTAL HEALTH RESEARCH INSTITUTE OF VICTORIA, Parkville, Victoria (AU)

(21) Appl. No.: 12/443,442

(22) PCT Filed: Oct. 23, 2007
(86) PCT No.: PCT/AU2007/001611

§ 371 Date: Nov. 10, 2009

Related U.S. Application Data
(60) Provisional application No. 60/853,572, filed on Oct. 23, 2006.

(51) Int. Cl. A61K 31/22; A61K 31/164; A61K 31/19; A61P 25/18; A61P 25/22; A61P 25/24; A61P 25/32; A61P 25/28

(52) U.S. Cl. 514/546; 514/625; 514/557

(57) ABSTRACT

The present invention relates generally to a method of treating a psychiatric or neuropsychiatric condition in a mammal with a combination therapy. More particularly, the present invention relates to a combination therapy comprising an antipsychotic agent and a compound that increases levels of glutathione in the body.

### FIELD OF THE INVENTION

The present invention relates generally to a method of treating a psychiatric or neuropsychiatric condition in a mammal with a combination therapy. More particularly, the present invention relates to a combination therapy comprising an antipsychotic agent and a compound that increases levels of glutathione in the body.

### BACKGROUND OF THE INVENTION

Bibliographic details of the publications referred to in the specification are collected at the end of the description.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

Mental illness such as schizophrenia, bipolar disorder, depression, affect a large number of the population. For example, schizophrenia is a severe mental illness which affects approximately one person in a hundred. Symptoms characterising schizophrenia include delusions (false beliefs of persecution, guilt, grandeur or being under outside control), hallucinations (visual or auditory) and thought disorder (speech which is difficult to follow or jumping from one subject to another with no logical connection). Secondary symptoms of schizophrenia include loss of drive, blunted emotions, social withdrawal and/or lack of insight.

The onset of schizophrenia usually occurs during adolescence or early adulthood, although it has been known to develop in older people. Onset may be rapid, with acute symptoms developing over several weeks, or it may be slow, developing over months or even years.

The causes of schizophrenia are not fully understood. However, during the last few years there has emerged a body of literature which supports an abnormality in oxidation homeostasis systemically and centrally in schizophrenia. The origin of this oxidative stress is still unknown. The brain in schizophrenia exhibits many chemical hallmarks of oxidative attack, in addition to indications of altered antioxidant defence. Any tissue under sustained radical attack may suffer a depletion of the key free radical/H₂O₂ scavenger in the brain, glutathione. Recently, reports have emerged that glutathione is indeed depleted in schizophrenia, and that the antioxidant enzymic activities related to glutathione metabolism are markedly perturbed. Do K Q et al. (2000), have reported a significant decrease (−27%) in the cerebrospinal fluid levels of glutathione in drug-free schizophrenia patients compared to controls. This decrease is consistent with the previously reported decrease in the levels of the glutathione metabolite gamma-glutamylglutamine in the cerebrospinal fluid of such patients (Do K Q et al., 1995). Furthermore, Do et al., (2000) also found a 52% decrease in glutathione levels in the medial prefrontal cortex of schizophrenia patients compared to controls, using a non-invasive proton magnetic resonance spectroscopy method.

Intriguingly, other aspects of the glutathione metabolic pathway are also perturbed in schizophrenia. Decreased peripheral glutathione peroxidase (GPx) activity has been described in schizophrenia patients (Abdalla D S et al., 1986), and the decrease correlates with increased brain atrophy (Buckman T D et al., 1987). Plasma GPx positively correlates with psychosis rating scored in schizophrenia patients on or off medication (Yao J K et al., 1999). GPx is the enzyme that catalyses the scavenging of H₂O₂ and other radicals by glutathione.

There is also some indirect evidence to suggest that depleted levels of glutathione may play a role in mood disorders such as depression and bipolar disorders, as well as substance use and autism.

To date, research has focused on the use of indirect means of overcoming the defects in glutathione metabolism such as increasing the efficiency of other radical scavenging systems. For example, Vitamin C, Vitamin E (alpha-tocopherol), alpha-lipoic acid supplements and also selenomethionione have been investigated. Currently, investigators are focusing on the use of Vitamins E and C (Yao et al., 1999, supra). Selenomethionione supplementation is well known to augment the activity of glutathione peroxidase (Duffield A J et al., 1999). Vitamin E and selenium combined supplementation has already been reported to provide beneficial effects in the treatment of the FALS transgenic mouse model (Gurney M E et al., 1996), demonstrating that the potential antioxidant benefits of such oral supplementation can also be transduced across the blood brain barrier in brain oxidation disorders. However, while being supportive of glutathione metabolism, in that these molecules can function as antioxidants, they are not the most efficient means of increasing glutathione levels in the brain.

Furthermore, many patients suffering mental disorders are medicated with antipsychotic drugs such as clozapine, haloperidol or risperidone. These drugs have many side effects including drug induced Parkinsonism, akathisia, tardive dyskinesia, diabetes, liver toxicity, cataracts, dry eyes, acute dystonias, tachycardia, hypotension, impotence, lethargy, dysphoria, seizures, hyperprolactinema and neuroleptic malignant syndrome. The side effects are drug dependent. For instance, atypical antipsychotics such as olanzapine appear to cause weight gain more readily than typical antipsychotics. As a result such atypical antipsychotics have been implicated in the onset of diabetes. Also, patients administered with clozapine regularly undergo blood checks as the drug is known to induce agranulocytosis, a condition where the number of white blood cells in the body may be dangerously reduced.

Accordingly, there is an ongoing need to develop methods of treating psychiatric and neuropsychiatric disorders that further increase glutathione levels, for instance, in the brain or blood, that do not exacerbate, and preferably reduce, the side effects caused by antipsychotic drugs.

In work leading up to the present invention, the inventors have determined that therapy with a combination of an antipsychotic drug and a compound that increases levels of glutathione, provides a reduction in the occurrence and/or severity of a mental illness such as schizophrenia, and may also reduce some of the side effects of the antipsychotic drug.

### SUMMARY OF THE INVENTION

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

One aspect of the present invention provides a method of treating a psychiatric or neuropsychiatric disorder comprising administering to a mammal a combination of an antipsychotic drug and a compound that increases glutathione levels in said mammal.

A further aspect of the present invention provides a method of reducing the side effects of an antipsychotic drug comprising administering to a mammal an antipsychotic drug in combination with a compound that increases glutathione levels in said mammal.

Another aspect of the present invention provides a pharmaceutical composition comprising an antipsychotic drug and a compound that increases glutathione levels.

Another aspect of the present invention provides a pharmaceutical composition comprising an antipsychotic drug and a glutathione precursor.

Yet another aspect of the present invention provides a use of an antipsychotic drug in the manufacture of a medicament for treatment of a psychiatric or neuropsychiatric disorder, wherein the antipsychotic drug is administered in combination with a compound that increases glutathione levels.

A further aspect of the present invention provides a use of a compound that increases glutathione levels in the manufacture of a medicament for treatment of a psychiatric or neuropsychiatric disorder, wherein the compound is administered in combination with an antipsychotic drug.

Yet a further aspect of the present invention provides a use of an antipsychotic drug and a compound that increases glutathione levels in the manufacture of a medicament for treating a psychiatric or neuropsychiatric disorder.

In yet another aspect of the invention there is provided a method of treating a psychiatric or neuropsychiatric disorder comprising administering to a mammal a combination of an antipsychotic drug and a glutathione precursor, or a pharmaceutically acceptable salt thereof.

In yet another aspect of the invention there is provided a method of treating a psychiatric or neuropsychiatric disorder comprising administering to a mammal a combination of an antipsychotic drug and a compound of formula (I):

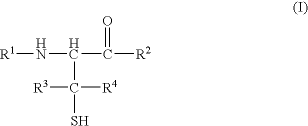

wherein
R¹ is selected from —C(O)C₁₋₄alkyl and —C(O)(CH₂)₂CH[C(O)R⁵]NHR⁶,
R² is selected from —OR⁷, —NH₂ and —NHCH₂C(O)R⁸,
R³ and R⁴ are independently selected from H and —C₁₋₄alkyl,
R⁵ is selected from —OH, —OC₁₋₄alkyl and NH₂,
R⁶ is selected from H, or C(O)C₁₋₄alkyl,
R⁷ is selected from H and C₁₋₄alkyl, and
R⁸ is selected from OH, —OC₁₋₄alkyl and NH₂,
and pharmaceutically acceptable salts thereof.

### BRIEF DESCRIPTION OF FIGURES

FIG. 1. Mean change in CGI-S from baseline over the study period. Severity is rated on a seven-point scale (1=normal to 7=extremely ill). *p<0.05 vs placebo, **p<0.01 vs placebo, PDV: post-discontinuation visit. P-values are from MMRM adjusted for baseline score and investigator.

FIG. 2. Proportion of participants with a score of 3 or less (improvement) on the CGI-I over the study period. *p<0.05, **p<0.01. P-values are from Fisher's exact test.

FIG. 3. Mean change in BAS from baseline over the study period. *p<0.05 vs placebo, PDV: post-discontinuation visit. P-values are from MMRM adjusted for baseline score and investigator.

FIG. 4. Adjusted effect size at week 24 compared to baseline for primary and secondary outcome measures. Data are mean effect size (Cohen's d statistic)±95% confidence intervals All analyses were adjusted for baseline and investigator using ANCOVA. *p<0.05 vs placebo, **p<0.01 vs placebo.

FIG. 5. Effects of NAC and placebo on outcome measures over the study period. Data are mean changes (±SEM) in scores from baseline at subsequent visits and at the post-discontinuation visit (PDV). *p<0.05 vs placebo, **p<0.01 vs placebo, ***p<0.005 vs placebo. P-values are from MMRM adjusted for baseline score and investigator.

FIG. 6. Adjusted effect size (MMRM) at week 24 compared to baseline for primary and secondary outcome measures. Data are mean effect size (Cohen's d statistic)±95% confidence intervals. MMRM adjusted for baseline score and investigator.

FIG. 7. NAC and NACA rescue striatal glutathione levels that are depleted by CHX. Data are means in SEM, N=5 in each group, readings done in triplicate.

FIG. 8. NAC and NACA rescue liver glutathione levels that are depleted by CHX. Data are means in SEM, N=5 in each group, readings done in triplicate.

### DETAILED DESCRIPTION OF THE INVENTION

The present invention is predicated, in part, on the determination that the administration of a combination of a compound that increases glutathione levels, in particular N-acetyl cysteine, and an antipsychotic drug can reduce the occurrence and/or severity of the symptoms of psychiatric or neuropsychiatric disorders, especially schizophrenia, and may also reduce the side effects associated with the antipsychotic drug. It is believed that the therapeutic effects of the increased levels of glutathione take place predominately in the central nervous system (ie the brain). It is however possible that the present invention acts by elevating peripheral glutathione levels, for instance, elevating glutathione levels in the blood.

Accordingly, in one aspect of the present invention there is provided a method of treating a psychiatric or neuropsychiatric disorder in a mammal comprising administering a combination of an antipsychotic drug and a compound that increases glutathione levels in said mammal.

In another aspect of the present invention there is provided a method of reducing the side effects of an antipsychotic drug comprising administering to a mammal an antipsychotic drug in combination with a compound that increases glutathione levels in said mammal.

The terms “neuropsychiatric disorder” and “psychiatric disorder” refers to mental disorders that may be treated with an antipsychotic drug. Neuropsychiatric disorders are a subclass of psychiatric disorders which deal with mental disorders attributable to diseases of the nervous system, such as, for example brain trauma, HIV or Lyme disease. Examples of psychiatric and neuropsychiatric disorders include schizophrenia, including childhood schizophrenia substance abuse (eg amphetamine induced psychosis), psychosis (including first episode psychosis), bipolar disorder, manic depression, major depression, affective disorder, schizophreniform or schizoaffective disorders, depression, psychotic depression, drug induced psychosis, delirium, autism, nausea, vertigo, inner ear infection (labyrithitis), chronic pain, palliative care (eg cancer pain), agonal agitation (ie end of life agitation), alcohol withdrawal syndrome, dementia induced psychosis, mood disorders and other psychotic disorders including first episode psychoses. Preferably, the present invention is directed to the treatment of schizophrenia.

In another embodiment the invention is directed to the treatment of bipolar disorder, major depression or first episode psychosis.

Suitable antipsychotic drugs include any drugs administered to reduce the occurrence and/or severity of symptoms such as psychotic episodes. Examples of antipsychotic drugs include, but are not limited to, clozapine, fluoxetine, olanzapine, symbyax (combination of olanzapine and fluoxetine), risperidone, haloperidol, droperidol, pimozide, quetiapine, chlorpromazine, amisulpride, fluphenazine, aripriprazole, flupenthixol, zuclopenthixol, trifluoperazine, valproate, lithium, ziprasidone, bifeprunox, norclozapine and tetrabenazine. Preferred antipsychotic drugs in respect of the present invention include clozapine, olanzapine, aripiprazole, quetiapine and ziprasadone.

Compounds that may increase glutathione levels in the body include glutathione and cysteine precursors as well as glutathione and cysteine themselves. Without limiting the present invention to any one theory or mode of action, glutathione is a tri-peptide containing a sulphydryl group which is widely distributed in living tissue. It is also known by the alternative name of α-glutamylcysteinylglycine or the abbreviation GSH. Glutathione is generally formed as a result of the actions of specific enzymes and not as a direct result of the usual processes of peptide synthesis, being transcription and translation of a nucleic acid molecule specifically encoding said peptide. Glutathione is a molecule of the formula HO₂CCH(NH₂)CH₂CH₂CONHCH(CH₂SH)CONHCH₂CO₂H. It should be understood that the regulation of a physiological process or pathway by a glutathione precursor is encompassed within the present invention. The first step in the synthesis of glutathione is the formation of a peptide linkage between the gamma-carboxyl group of glutamate and the amino group of cysteine to form gamma-glutamyl-cysteine. This is catalysed by gamma-glutamylcysteine synthetase. Formation of this peptide bond requires activation of the gamma-carboxyl group, which activation is provided by ATP. The resulting molecule is an intermediate which is then attacked by the amino group of cysteine. In this second step, which is catalysed by glutathione synthetase, ATP activates the carboxyl group of cysteine to enable it to condense with the amino group of glycine. Accordingly, glutathione is a molecule which is formed subsequently to the actions of enzymes on the rate limiting precursor cysteine. Glutathione cycles between a reduced thiol form (GSH) and an oxidised form (GSSG) in which two tripeptides are linked by a disulfide bond.

In this regard, reference to a “glutathione precursor” should be understood as a reference to any molecule from which glutathione can be directly or indirectly derived. The subject molecule may be naturally or non-naturally occurring. Modification of a molecule in a single step to form glutathione is an example of glutathione being directly derived from a precursor. Modification of a molecule to form an “intermediate” molecule, which intermediate molecule undergoes further modification to form glutathione is an example of glutathione being indirectly derived from the subject precursor.

Cysteine is a naturally occurring precursor from which glutathione is indirectly derived. Accordingly, cysteine and cysteine precursors are glutathione precursors according to the present invention. Specifically, cysteine is catalysed to form gamma-glutamyl cysteine prior to catalysis of this molecule to take up glycine and thereby form glutathione.

Glutathione precursors also include molecules that are non-naturally occurring and that produce an intermediate molecule in vivo that is then used in the biosynthesis of glutathione, such molecules include, but are not limited to, cysteine derivatives such as N-acetyl cysteine and N-acetyl cysteine amide.

In a preferred embodiment the compound that increases glutathione levels is a glutathione precursor.

In an even more preferred embodiment the compound that increases glutathione levels is a compound of formula (I):

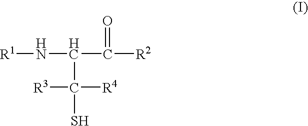

wherein
R¹ is selected from —C(O)C₁₋₄alkyl and —C(O)(CH₂)₂CH[C(O)R⁵]NHR⁶,
R² is selected from —OR⁷, —NH₂ and —NHCH₂C(O)R⁸,
R³ and R⁴ are independently selected from H and —C₁₋₄alkyl,
R⁵ is selected from —OH, —OC₁₋₄alkyl and NH₂,
R⁶ is selected from H, or C(O)C₁₋₄alkyl,
R⁷ is selected from H and C₁₋₄alkyl, and
R⁸ is selected from OH, —OC₁₋₄alkyl and NH₂,
and pharmaceutically acceptable salts thereof.

In preferred embodiments of formula (I) at least one of the following applies:

R¹ is —C(O)CH₃, —C(O)(CH₂)₂CH(CO₂H)NHC(O)CH₃, —C(O)(CH₂)₂CH(CO₂CH₃)NHC(O)CH₃, —C(O)(CH₂)₂CH(CO₂CH₂CH₃)NHC(O)CH₃ or —C(O)(CH₂)₂CH(CONH₂)NHC(O)CH₃; especially —C(O)CH₃, —C(O)(CH₂)₂CH(CO₂H)NHC(O)CH₃, —C(O)(CH₂)₂CH(CO₂CH₂CH₃)NHC(O)CH₃ or —C(O)(CH₂)₂CH(CONH₂)NHC(O)CH₃; more especially —C(O)CH₃;
R² is —OH, —OCH₃, —OCH₂CH₃, —NH₂, —NHCH₂CO₂H, —NHCH₂CO₂CH₃, —NHCH₂CO₂CH₂CH₃, or —NHCH₂CONH₂; especially —OH, —OCH₂CH₃, —NH₂, —NHCH—₂CO₂H, —NHCH₂CO₂CH₂CH₃ or —NHCH₂CO₂NH₂; more especially —OH or —NH₂.
R³ is H or —CH₃, especially H; and
R⁴ is H or —CH₃, especially H.

Preferred compounds of formula (I) include:
N-acetyl cysteine,
N-acetyl cysteine amide,
N-acetyl cysteine ethyl ester,
N-acetyl β,β-dimethyl cysteine ether ester (N-acetylpenicilamine ethyl ester),
N-acetyl β,β-cysteine (N-acetyl penicilamine),
Glutathione ethyl ester,
N-acetyl glutathione ethyl ester,
N-acetyl glutathione,
N-acetyl α-glutamyl ethyl ester cysteinyl glycyl ethyl ester (N-acetyl(β-ethyl ester)glutathione ethyl ester),
N-acetyl α-glutamyl ethyl ester cysteinyl glycine (N-acetyl(β-ethyl ester)glutathione),
γ-glutamyl cysteine ethyl ester,
N-acetyl glutathione amide,
N-acetyl β,β-dimethyl cysteine amide,
N-acetyl β-methyl cysteine amide, and
N-acetyl cysteine glycine amide.

In another preferred embodiment the compound that increases glutathione levels is a compound of formula (II):

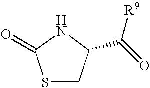

where R⁹ is selected from OH, OC₁₋₆ alkyl, NH₂ (C₁₋₆ alkyl) and N(C₁₋₆ alkyl)₂. A preferred compound of formula (II) is procysteine.

As used herein the term “alkyl” refers to a saturated straight or branched hydrocarbon chain. The alkyl group may have a specified number of carbon atoms, for examples, C₁₋₄alkyl is a straight or branched hydrocarbon chain having 1, 2, 3 or 4 carbon atoms. Examples of alkyl groups include, but are not limited to, methyl, ethyl, propyl, isopropyl, butyl, 2-methyl-propyl and tent-butyl.

The compounds that increase glutathione levels may be in the form of pharmaceutically acceptable salts. It will be appreciated however that non-pharmaceutically acceptable salts also fall within the scope of the invention since these may be useful as intermediates in the preparation of pharmaceutically acceptable salts or may be useful during storage or transport. Suitable pharmaceutically acceptable salts include, but are not limited to, salts of pharmaceutically acceptable inorganic acids such as hydrochloric, sulphuric, phosphoric, nitric, carbonic, boric, sulfamic, and hydrobromic acids, or salts of pharmaceutically acceptable organic acids such as acetic, propionic, butyric, tartaric, maleic, hydroxymaleic, fumaric, maleic, citric, lactic, mucic, gluconic, benzoic, succinic, oxalic, phenylacetic, methanesulphonic, toluenesulphonic, benezenesulphonic, salicyclic sulphanilic, aspartic, glutamic, edetic, stearic, palmitic, oleic, lauric, pantothenic, tannic, ascorbic and valeric acids.

Base salts include, but are not limited to, those formed with pharmaceutically acceptable cations, such as sodium, potassium, lithium, calcium, magnesium, ammonium and alkylammonium.

Basic nitrogen-containing groups may be quarternised with such agents as lower alkyl halide, such as methyl, ethyl, propyl, and butyl chlorides, bromides and iodides; dialkyl sulfates like dimethyl and diethyl sulfate; and others.

It will also be recognised that compounds of the invention may possess asymmetric centres and are therefore capable of existing in more than one stereoisomeric form. The invention thus also relates to compounds in substantially pure isomeric form at one or more asymmetric centres eg., greater than about 90% ee, such as about 95% or 97% ee or greater than 99% ee, as well as mixtures, including racemic mixtures, thereof. Such isomers may be prepared by asymmetric synthesis, for example using chiral intermediates, or by chiral resolution.

The side effects of an antipsychotic drug that may be reduced or inhibited by the method of the present invention includes extra pyramidal side effects (ie various movement disorders) such as drug induced Parkinsonism, acute dystonias, tachycardia, hypotension, impotence, lethargy, akathisia, seizures, hyperprolactinema and tardive dyskinesia. Other side effects include diabetes, liver toxicity, cataracts, dry eyes, dysphoria, and neuroleptic malignant syndrome.

The term “combination” as used herein refers to the administration of the antipsychotic drug and the compound that increases glutathione levels in the central nervous system simultaneously in a single composition or separately or sequentially in different compositions. The two components are administered so that they are biologically active, at least in part, at the same time. In some cases, one of the components may require multiple doses while the other component requires a single dose per day. In other cases, each component requires multiple doses per day to maintain an effective dose of each component so they are, at least partially, active at the same time.

The term “mammal” as used herein includes humans, primates, livestock animals (eg. sheep, pigs, cattle, horses, donkeys), laboratory test animals (eg. mice, rabbits, rats, guinea pigs), companion animals (eg. dogs, cats) and captive wild animals (eg. foxes, kangaroos, deer). Preferably, the mammal is human or a laboratory test animal. Even more preferably, the mammal is a human.

Reference to “treatment” is to be considered in its broadest context. The term “treatment” does not necessarily imply that a subject is treated until total recovery or that the treatment provides a complete recovery. Accordingly, treatment includes amelioration of symptoms or the onset of symptoms of a particular condition or disorder or reduction in the severity or duration of a particular condition or symptom. Treatment may also include a reduction in side effects caused by one of the components in the combination therapy. Accordingly, the term “treatment” is intended to include both prophylactic treatment as well as therapeutic treatments.

The method of the present invention preferably facilitates the psychiatric or neuropsychiatric disorder being reduced, retarded or otherwise inhibited. Reference to “reduced, retarded or otherwise inhibited” should be understood as a reference to inducing or facilitating the partial or complete inhibition of any one or more causes or symptoms of the neuropsychiatric disorder. In this regard, it should be understood that conditions such as psychiatric or neuropsychiatric disorders are extremely complex comprising numerous physiological events which often occur simultaneously. It should be understood that the present invention contemplates both relieving any one or more symptoms of the disorder (for example, relieving one or more psychosis events) or facilitating retardation or cessation of the cause of the disorder (for example, reducing oxidative stress thereby minimising any further neuronal damage). In some methods of the present invention, the side effects of antipsychotic drugs are reduced, retarded or otherwise inhibited. It should be understood that reference to “reduced, retarded or otherwise inhibited” includes inducing or facilitating the partial or complete inhibition of one or more side effects caused by the antipsychotic drug, especially movement disorders.

Administration of the antipsychotic drug and compound that increases glutathione levels, may be performed simultaneously, separately or sequentially and in any convenient manner. An “effective amount” means an amount of each component necessary at least partly to attain the desired response, or to delay the onset or inhibit progression or halt altogether one or more symptoms, or the progression of a particular condition being treated or an amount of each component required to delay the onset of, inhibit the progression of or halt altogether one or more side effects of the antipsychotic drug. The amount varies depending upon the health and physical condition of the individual to be treated, the taxonomic group of individual to be treated, the degree of protection desired, the formulation of the composition or compositions, the assessment of the medical situation, and other relevant factors. It is expected that the amount will fall in a relatively broad range that can be determined through routine trials.

An effective amount of antipsychotic drug may be an amount which is normally provided when the antipsychotic drug is administered in the absence of the compound that increases glutathione levels. For example, clozapine is typically administered at 12.5-900 mg, or more usually 100-300 mg, three times per day. Valproate is typically administered at 1000-2500 mg/day in three divided doses. Lithium is typically administered at 0.5-1 g per day in divided doses such as twice or three times per day. Alternatively, the antipsychotic drug may be administered in amounts less than normally provided when the antipsychotic drug is administered in the absence of the compound that increases glutathione levels.

An effective amount of the compound that increases glutathione levels may be adjusted to provide the optimum therapeutic response. For example, a dosage of 10 mg to 150 mg per kg of body weight per day. The effective amount may be administered as a single dose or as several divided doses daily, weekly, monthly or at other suitable time intervals, or the dose may be proportionally reduced as indicated by the exigencies of the situation. The compound may be administered in a convenient manner such as by the oral, intravenous (where water soluble), intraperitoneal, intramuscular, subcutaneous, intradermal or suppository routes or implanting (e.g. using slow release of molecules). Preferably the compound is administered orally and dosages of 0.1-10 grams per day. More particularly the dosage is 1-5 grams per day, especially about 2 grams per day.

The two compounds may be administered in a single composition or may be administered in separate compositions. If administered in a single composition or separate compositions, oral administration of both components is preferred. However, if administered separately, the components may be administered by the same or different routes. If administered sequentially, the components may be administered in any order.

In an embodiment the antipsychotic drug and the compound that increases glutathione levels may be presented in a single composition, for instance, a capsule. For instance, a single composition of the present invention may involve a capsule containing clozapine and NAC therein. An example of such a composition may be 150 mg of clozapine and 600 mg of NAC in a capsule. It is envisaged that a 300 mg clozapine/700 mg NAC single dose could also be possible. Such doses may be taken three times per day.

In another embodiment 0.25-16 mg/day of risperidone may be taken with the compound that increases glutathione levels either as a single composition or as separate doses. Typically, 0.5-8.0 mg/per day of risperidone may be used.

The compound that increases glutathione levels may also be administered in the form of a prodrug. The term “prodrug” is used in its broadest sense and encompasses those derivatives that are converted in vivo to the compounds of the invention. Such derivatives would readily occur to those skilled in the art, and include N-α-acyloxy amides, N-(acyloxyalkoxy carbonyl) amine derivatives and α-acyloxyalkyl esters of phenols and alcohols. A prodrug may include modifications to one or more of the functional groups of a compound of the invention.

The term “prodrug” also encompasses the combination of lipids with the compounds of the invention. The presence of lipids may assist in the translocation of the compounds across a cellular membrane and into a cell cytoplasm or nucleus. Suitable lipids include fatty acids which may be linked to the compound by formation of a fatty acid ester. Preferred fatty acids include, but are not limited to, lauric acid, caproic acid, palmitic acid and myristic acid.

The phrase “a derivative which is capable of being converted in vivo” as used in relation to another functional group includes all those functional groups or derivatives which upon administration into a mammal may be converted into the stated functional group. Those skilled in the art may readily determine whether a group may be capable of being converted in vivo to another functional group using routine enzymatic or animal studies.

The antipsychotic drugs useful in the combination therapy may be obtained commercially or prepared by known synthetic methods. The compounds that increase glutathione levels may also be commercially available or may be synthesised by known methods. For example, N-acetyl cysteine (NAC) may be obtained commercially [Aldrich 616-91-1] or may be prepared from cysteine by N-acetylation. For example, N-acetylation may be effected by reacting a cysteine in which the carboxy group is optionally protected with acetylanhydride in the presence of a base. Other compounds of formula (I) may be prepared by known procedures such as acetylation, esterification and amide bond formation. The reactions may be directed to particular sites and sensitive groups prevented from reaction by use of protecting groups well known in peptide synthesis. Compounds of formula (II) may be prepared based on known chemistry for preparing substituted oxothiazolidines. The synthesis of a number of compounds of formula (I) include N-acetyl cysteine amide, N-acetyl cysteine ethyl ester, N-acetyl β,β-dimethyl cysteine ether ester (N-acetylpenicilamine ethyl ester), N-acetyl β,β-cysteine (N-acetyl penicilamine), Glutathione ethyl ester, N-acetylglutathione ethyl ester, N-acetyl glutathione, N-acetyl α-glutamyl ethyl ester cysteinyl glycyl ethyl ester (N-acetyl(β-ethyl ester)glutathione ethyl ester), N-acetyl α-glutamyl ethyl ester cysteinyl glycine (N-acetyl(β-ethyl ester)glutathione), N-acetyl glutathione amide, N-acetyl β,β-dimethyl cysteine amide, N-acetyl β-methyl cysteine amide, and N-acetyl cysteine glycine amide, is given in U.S. Pat. No. 6,420,429.

Although each component in the combination therapy may be administered alone or as a mixture, preferably administration of each component is in the form of a single pharmaceutical composition or each component may be administered as separate pharmaceutical compositions. Each pharmaceutical composition whether containing both components or one component may include one or more pharmaceutically acceptable carriers.

In one aspect of the present invention there is provided a pharmaceutical composition comprising an antipsychotic drug and a compound that increases glutathione levels, optionally with one or more pharmaceutically acceptable carriers.

The carrier(s) must be “acceptable” in the sense of being compatible with the other ingredients of the composition and not deleterious to the recipient thereof.

Pharmaceutical formulations include those suitable for oral, rectal, nasal, topical (including buccal and sub-lingual), vaginal or parenteral (including intramuscular, sub-cutaneous and intravenous) administration or in a form suitable for administration by inhalation or insufflation. Each component of the invention, together with a conventional adjuvant, carrier, excipient, or diluent, may thus be placed into the form of a single or separate pharmaceutical compositions and unit dosages thereof, and in such form may be employed as solids, such as tablets or filled capsules, or liquids such as solutions, suspensions, emulsions, elixirs, or capsules filled with the same, all for oral use, in the form of suppositories for rectal administration; or in the form of sterile injectable solutions for parenteral (including subcutaneous) use. Such pharmaceutical compositions and unit dosage forms thereof may comprise conventional ingredients in conventional proportions, with or without additional active compounds or principles, and such unit dosage forms may contain any suitable effective amount of the active ingredient commensurate with the intended daily dosage range to be employed. The components of the present invention can be administered in a wide variety of oral and parenteral dosage forms especially oral dosage forms. It will be obvious to those skilled in the art that the following dosage forms may comprise, as the active components, either the components of the invention or pharmaceutically acceptable salts of the components of the invention.

For preparing pharmaceutical compositions from the components of the present invention, either together or separately, pharmaceutically acceptable carriers can be either solid or liquid. Solid form preparations include powders, tablets, pills, capsules, cachets, suppositories, and dispersible granules. A solid carrier can be one or more substances which may also act as diluents, flavouring agents, solubilizers, lubricants, suspending agents, binders, preservatives, tablet disintegrating agents, or an encapsulating material.

For instance, in an embodiment a solid preparation may include at least one other antioxidant (ie preservative). Suitable antioxidants are known in the art and include ascorbate or metabisulfite. This is especially preferred to prevent oxidation of the free sulfhydryl group on the compound of formula (I) (for instance, NAC) or precursors thereof. Another way of preventing oxidation of such compounds is to formulate such that the presence of oxygen within the formulation is minimised or prevented. This may include, for instance, airtight encapsulation or the use of a sealed gelatin capsule.

In powders, the carrier is a finely divided solid which is in a mixture with the finely divided active components either together or separately.

In tablets, the active components are together or separately mixed with the carrier having the necessary binding capacity in suitable proportions and compacted in the shape and size desired.

The powders and tablets preferably contain from five or ten to about seventy percent of the active components. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the components, either together or separately, with encapsulating material as carrier providing a capsule or capsules in which the active components, with or without carriers, is surrounded by a carrier, which is thus in association with it. Similarly, cachets and lozenges are included. Tablets, powders, capsules, pills, cachets, and lozenges can be used as solid forms suitable for oral administration.

For preparing suppositories, a low melting wax, such as admixture of fatty acid glycerides or cocoa butter, is first melted and the active component is dispersed homogeneously therein, as by stirring. The molten homogenous mixture is then poured into convenient sized molds, allowed to cool, and thereby to solidify.

Formulations suitable for vaginal administration may be presented as pessaries, tampons, creams, gels, pastes, foams or sprays containing in addition to the active components, either together or separately, such carriers as are known in the art to be appropriate.

Liquid form preparations include solutions, suspensions, and emulsions, for example, water or water-propylene glycol solutions. For example, parenteral injection liquid preparations can be formulated as solutions in aqueous polyethylene glycol solution.

The components according to the present invention may thus be formulated together or separately for parenteral administration (e.g. by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, and may contain formulatory agents such as suspending, stabilising and/or dispersing agents. Alternatively, the active components may be in powder form, obtained by aseptic isolation of sterile solid or by lyophilisation from solution, for constitution with a suitable vehicle, e.g. sterile, pyrogen-free water, before use.

Aqueous solutions suitable for oral use can be prepared by dissolving the active components, together or separately, in water and adding suitable colorants, flavours, stabilizing and thickening agents, as desired.

Aqueous suspensions suitable for oral use can be made by dispersing the finely divided active components, together or separately in water with viscous material, such as natural or synthetic gums, resins, methylcellulose, sodium carboxymethylcellulose, or other well known suspending agents.

Also included are solid form preparations which are intended to be converted, shortly before use, to liquid form preparations for oral administration. Such liquid forms include solutions, suspensions, and emulsions. These preparations may contain, in addition to the active components, either together or separately, colorants, flavours, stabilizers, buffers, artificial and natural sweeteners, dispersants, thickeners, solubilizing agents, and the like.

For topical administration to the epidermis the components, either together or separately, may be formulated as ointments, creams or lotions, or as a transdermal patch. Ointments and creams may, for example, be formulated with an aqueous or oily base with the addition of suitable thickening and/or gelling agents. Lotions may be formulated with an aqueous or oily base and will in general also contain one or more emulsifying agents, stabilising agents, dispersing agents, suspending agents, thickening agents, or colouring agents.

Solutions or suspensions are applied directly to the nasal cavity by conventional means, for example with a dropper, pipette or spray. The formulations may be provided in single or multidose form. In the latter case of a dropper or pipette, this may be achieved by the patient administering an appropriate, predetermined volume of the solution or suspension. In the case of a spray, this may be achieved for example by means of a metering atomising spray pump. To improve nasal delivery and retention the components according to the invention may be encapsulated with cyclodextrins, or formulated with their agents expected to enhance delivery and retention in the nasal mucosa.

Administration to the respiratory tract may also be achieved by means of an aerosol formulation in which one or both of the components is provided in a pressurised pack with a suitable propellant such as a chlorofluorocarbon (CFC) for example, dichlorodifluoromethane, trichlorofluoromethane, or dichlorotetrafluoroethane, carbon dioxide, or other suitable gas. The aerosol may conveniently also contain a surfactant such as lecithin. The dose of component(s) may be controlled by provision of a metered valve.

Alternatively the active components, together or separately, may be provided in the form of a dry powder, for example a powder mix of the compound in a suitable powder base such as lactose, starch, starch derivatives such as hydroxypropylmethyl cellulose and polyvinylpyrrolidone (PVP).

Conveniently the powder carrier will form a gel in the nasal cavity. The powder composition may be presented in unit dose form for example in capsules or cartridges of, e.g., gelatin, or blister packs from which the powder may be administered by means of an inhaler.

In formulations intended for administration to the respiratory tract, including intranasal formulations, the compound will generally have a small particle size for example of the order of 1 to 10 microns or less. Such a particle size may be obtained by means known in the art, for example by micronization.

When desired, formulations adapted to give slow or sustained release of the active components may be employed. Such formulations are known in the art and so too are the slow or sustained release excipients. Other techniques used to obtain slow or sustained release such as compaction and the use of an enteric coating is also envisaged. Other formulations amenable to slow or sustained release such as a subcutaneous implant as a rod, capsule or bar, or a transdermal patch are also contemplated.

The pharmaceutical preparations are preferably in unit dosage forms. In such form, the preparation is subdivided into unit doses containing appropriate quantities of the active components, either together or separately. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

Liquids or powders for intranasal administration, tablets or capsules for oral administration and liquids for intravenous administration are preferred compositions.

The invention will now be described with reference to the following Example which illustrate some preferred aspects of the present invention. However, it is to be understood that the particularity of the following description of the invention is not to supersede the generality of the preceding description of the invention.

### Example 1

### N-acetyl cysteine as a glutathione Precursor for Schizophrenia

There is preclinical evidence of a reduction in antioxidant defences in schizophrenia, particularly a reduction in glutathione, which is a primary endogenous antioxidant defence. N-acetyl cysteine (NAC) is a bioavailable precursor of glutathione. This Example is a randomised double blind multicentre placebo controlled trial of the use of NAC in combination with an antipsychotic agent in the treatment of schizophrenia. A total of 140 individuals were randomised to receive 2 g daily of NAC in two divided doses, or placebo, together with their normal dosage of antipsychotic agent.

### Methods

### Study Design

Participants were assigned randomly and consecutively to treatment with NAC or placebo in a double blind fashion. The randomisation log was generated by an independent individual using the traditional coin tossing method. The investigators and clinicians remained blind until data analysis was completed. The person generating the randomisation schedule was not involved in participant interviews, either for participant eligibility or outcome measurements.

All participants remained on their usual antipsychotic medication for the duration of the trial. The dose of the primary therapy was monitored. Participants were recruited through advertisements, referral by case clinicians and through database screening. All participants provided written informed consent as part of complying with the protocol.

### Dose Rationale

NAC was purchased from Zambon, Italy. Purity was 99.8% as determined by HPLC. Encapsulation of the active compound and the inert placebo was performed by DFC Thompson, Sydney, Australia. NAC has a distinctive odor. In order to maintain the blind, bottles were sealed, dispensed by pharmacy, and returned to pharmacy so that the investigators did not have the opportunity to see them. Accordingly, pill counts were done by the pharmacy. Participants were seen individually and had no opportunity to compare reports.

All randomised participants received two NAC (500 mg) capsules twice daily, to a total dose of 2 g daily, or matching placebo capsules. Human dosing can be up to 5 g/day without adverse effects (Louwerse E. S. et al., 1995). A daily dose that is within the range of efficacy in published clinical trials (Adair J. C. et al., 2001, Van Schooten F. J. et al., 2002, and Behr J, et al., 2002) was selected. Once per day dosing is desirable in the drug treatment of schizophrenia, where medication compliance is known to be a challenge in management. However, steady state plasma levels cannot be achieved with a once daily oral dose of NAC as the plasma half-life is only 2-3 hours (Holdiness M. R., 1991 and Kelly G. S., 1998). While a twice-daily (BID) dosing regimen would also not be expected to achieve steady state plasma levels, dose intervals beyond BID were considered to markedly increase the risk of non-compliance in this population.

### Inclusion and Exclusion Criteria

To be included, the patients were required to meet Diagnostic and Statistical Manual of Mental Disorders, fourth edition (DSM-IV) (Association AP, 1994) criteria for schizophrenia and have a PANSS total score of ≧55, or at least two items in the positive and/or negative items being >3, or have a CGI-S≧3. They needed to have the capacity to consent to the study, and be aged between 18 and 65. Both inpatients and outpatients were eligible. Participants needed to be currently taking an antipsychotic agent and to be utilising effective contraception if female and of childbearing age. Exclusion criteria included patients with abnormal renal, hepatic, thyroid or haematological findings, patients with a systemic medical disorder including asthma, allergies or any history of bronchospasm, respiratory insufficiency and recent gastrointestinal ulcers and females who were positive on pregnancy screening testing at baseline. Participants who were taking a mood stabiliser (e.g. lithium, valproate and carbamazepine) were excluded as were those currently taking drugs that are known to prevent GSH depletion (500+mg of NAC/day, 200+ug of selenium/day or 500+IU of Vitamin E/day). Participants who had a prior adverse reaction to NAC or any component of the preparation, or who were unable to comply with the requirements of informed consent or the treatment protocol were also excluded.

### Participant Evaluation

Withdrawal from the trial occurred if participants ceased taking their trial medication for seven consecutive days, or ceased effective contraception, or became pregnant. A change in primary antipsychotic from one medication to another required participants to be withdrawn from the study. Similarly, the addition of a mood stabiliser required withdrawal of the participant. Dose changes to existing medications were not an exclusion criterion, and this was monitored. Participants on psychoactive medications for other indications (including antidepressants) had to have been on those agents for at least 1 month prior to randomisation. All participants gave written informed consent at baseline. Participants were withdrawn from the study if they withdrew consent or developed serious adverse events associated with the study drug. Discontinuation due to adverse events was either at the request of the participant or at the discretion of the investigator. The trial was approved by each participating research and ethics committee (Barwon Health, Southwest Area Mental Health Service, Bendigo Health, Ballarat Health, all in Victoria, Australia, and University of Lausanne, Switzerland), and was conducted according to GCP guidelines.

A summary of screening, enrolment and reasons for withdrawal is set out in Table 1.

TABLE 1
The Consort E-Flowchart-N-acetyl cysteine in schizophrenia trial

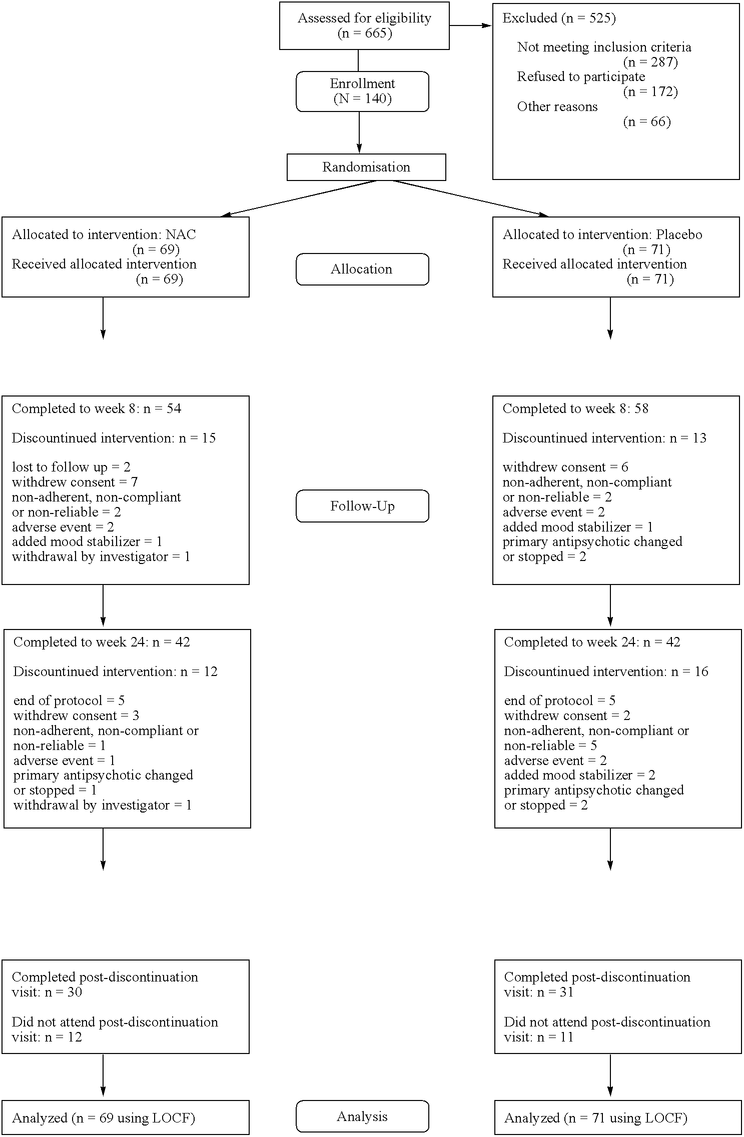

### Primary and Secondary Endpoints

Participants were assessed at baseline using a structured clinical interview (MINI, DSM-IV). The primary outcome measures used rating scales for psychotic illness: the Positive and Negative Symptom Scale (PANSS) and the Clinical Global Impression (CGI) improvement (CGI-I) and severity (CGI-S) scales. Secondary measures included the Global Assessment of Functioning Scale (GAF), the Social and Occupational Functioning Assessment Scale (SOFAS). In addition, extrapyramidal adverse effects were appraised using the Abnormal Involuntary Movements Scale (AIMS), the Simpson-Angus Scale (SAS) and the Barnes Akathisia Scale (BAS). Tolerability of treatment was assessed by endorsement scores on a checklist of 44 somatic items. The assessments were performed by blinded investigators whom received training to optimise reliability prior to the study. These scales were repeated two weekly for the first 8 weeks (“acute phase” treatment) or on the day of study termination if the participant withdrew prior to 8 weeks. Treatment continued from 8 weeks, with four-weekly evaluations to a total of 24 weeks, whereupon the NAC or placebo was stopped. A post-discontinuation follow-up visit was held 4 weeks (±2 weeks) after trial completion to determine any change in participant status after treatment discontinuation. An improvers analysis was performed on subjects with a CGI-I score of ≦3 at any 4 or more visits. While plasma glutathione levels were not assayed in this cohort, a parallel study determined that NAC at this dose significantly increased plasma glutathione.

A complete physical as well as a neurological examination was performed at baseline. Adverse events were tabulated. Routine laboratory investigations were carried out to assess renal, thyroid, haematological and hepatic function at baseline and at week 8. Blood pressure, pulse, and weight were monitored at each visit.

All randomized participants with at least one post baseline assessment were included in the analysis. Randomization occurred at Visit 1. Endpoint was defined as the last post-baseline value obtained for a patient for a given measure during the treatment phase. For patients who completed, endpoint corresponded to the Visit 9 (week 24) observation. For patients who discontinued early, endpoint corresponded to their last observation carried forward (LOCF).

### Statistical Analysis

Analysis was performed by an external consultant statistician, who was blind to treatment assignment, using SAS version 8.2 for Windows (SAS Institute, Cary, N.C.) on a clean and locked database. All analyses were conducted in accordance with the International Conference on Harmonization E9 statistical principles (International Conference on Harmonisation: Guidance on Statistical Principles for Clinical Trials, 1997). The primary analysis was performed according to the intention-to-treat principle and assessed average treatment group differences from baseline to visit 9 (week 24). This analysis examined the longitudinal profile of all the outcome measures in the study, and is a likelihood based mixed-effects model, repeated measures approach (MMRM). The MMRM model included the fixed, categorical effects of treatment, investigator, visit, and treatment-by-visit interaction, as well as the continuous, fixed covariates of baseline score and baseline score-by-visit interaction. The MMRM includes all available data at each time point (Mallinckrodt C. et al., 2004). MMRM analysis for improvers (subjects with a CGI-I score of ≦3 at any 4 or more visits) was performed for all outcomes to ascertain what components comprised the clinical improvement.

Analysis of covariance (ANCOVA) was used to also compare differences between treatment means in changes from baseline to endpoint. For this analysis and for those who discontinued early, endpoint corresponded to their last observation carried forward (LOCF). The ANCOVA model included the fixed, continuous covariate of baseline score as well as the categorical fixed effects of treatment, investigator and treatment-by-investigator interaction. Treatment-by-investigator interaction was tested at the 0.10 level. The secondary analysis was conducted on all other outcome measures in the same way as the primary analysis.

Results from the analysis of dichotomous data are presented as proportions, with 95% confidence interval, and Fisher's Exact p-value where appropriate. Non parametric statistics were used when assumptions for parametric methods were violated.

Kaplan Meier estimates and the log-rank test and the Wilcoxon-Breslow-Gehan test were used to evaluate time to all cause discontinuation. Effect sizes (Cohen's d) were calculated as the least square mean change from baseline to endpoint score in the outcome measure of the NAC group and the placebo group after adjusting for baseline score, investigator, treatment and treatment-by-investigator interaction where appropriate.

Effect sizes were calculated as the least square mean change from baseline to endpoint score in the outcome measure of the treatment group (NAC) and the control group (placebo) after adjusting for baseline score, investigator, treatment and treatment-by-investigator interaction where appropriate. The difference between these two scores was then divided by the square root of the pooled estimate of the standard deviation. As computed, a positive result would indicate that NAC favoured placebo; conversely, a negative result would indicate that placebo favoured NAC. Higher effect sizes indicate greater separation between treatment groups. Applying Cohen's guidelines (Cohen J., 1988), an effect size of 0.2-0.4 is considered a small effect, 0.5-0.7 is considered a medium effect and ≧0.8 is considered a large effect. For example, in a group of patients with treatment-resistant schizophrenia, switching from treatment with typical antipsychotic medication to optimal clozapine treatment was associated with a medium effect size (0.5) for improvement of specific positive and negative symptoms (Pickar D. and Bartko J., 2003). Samples of 40 to 100 patients are recommended for studies of drug augmentation in schizophrenia, on the basis of effect sizes of 0.5 to 0.8 (Anil Yagcioglu A. et al., 2005, Stern R. et al., 1997, Taylor C. et al., 2001 and Henderson D. and Goff D., 1996).

All tests of treatment effects were conducted using a two-sided alpha level of 0.05 and 95% confidence intervals were presented. No adjustments for multiple comparisons were made for this study. The term significant in this report indicates statistical significance (P≦0.05).

A summary of the baseline characteristics of participants is set out in Table 2.

TABLE 2
Baseline Characteristics of Participants

| | Placebo | | All |
|---|---|---|---|
| | Group | NAC Group | Participants |
| Characteristic* | (n = 71) | (n = 69) | (n = 140) |
| Age^{a}-yrs | 36.1 ± 11.7 | 37.2 ± 10.1 | 36.6 ± 10.91 |
| Male sex^{b}-no. (%) | 50 (70) | 48 (70) | 98 (70) |
| Duration of illness^{a}-yrs | 12.1 ± 9.6\hat{ } | 12.4 ± 8.2^{§} | 12.2 ± 8.9^{#} |
| Admission frequency | 1.0 (0.0-7.0) | 1.0 (0.0-7.0) | 1.0 (0.0-7.0) |
| score^{c}-median (range)^{‡} | | | |
| Smoking^{b}-Number of | 49 (69) | 46 (66) | 95 (68) |
| participants (%) | | | |
| Alcohol use^{b}-Number of | 41 (58) | 33 (48) | 74 (53) |
| participants (%) | | | |
| Substance use^{b}-Number | 13 (18) | 9 (13) | 22 (16) |
| of participants (%) | | | |
| Prior suicide attempt^{b}-Number | 4 [symbol] | 5^{¥} | 9⁺ |
| of participants | | | |

*Plus-minus values are means ± SD. unless otherwise noted. Differences between the NAC and placebo groups were not statistically significant (p ≦ 0.05) based on ^{a}two sample t-test (equal variance), ^{b}Fisher's exact test, or ^{c}Kruskall-Wallis analysis.
\hat{ }The data were obtained from 67 participants
^{§}The data were obtained from 64 participants
^{#}The data were obtained from 131 participants [symbol]
The data were obtained from 70 participants
^{¥}The data were obtained from 68 participants
⁺The data were obtained from 138 participants
^{‡}Admissions data were scored on the basis of 1 = 1 admission, 2 = 2 admissions, 3 = 3 admissions, 4 = 4 admissions, 5 = 5 admissions, 6 = 6-10 admissions, 7 = more than 10 admissions

TABLE 3
Primary and Secondary Outcome Measures at Baseline, and Change at Week 8 and Week 24

| | Within Placebo Group | | | Within NAC Group | |
|---|---|---|---|---|---|
| | | Week 8 | Week 24 | | Week 8 |
| Outcome | Baseline | Mean Overall^{b} | Mean Overall^{b} | Baseline | Mean Overall^{b} Change |
| Measure | Mean (SD) | Change (95% CI) | Change (95% CI) | Mean (SD) | (95% CI) |
| CGI-S | 4.0 (0.83) | −0.08 (−0.24, 0.08) | −0.03 (−0.23, 0.17) | 3.9 (0.89) | −0.32 (−0.48, −0.15)** |
| CGI-I^{c} | N/A | 3.2 (3.0, 3.5) | 3.5 (3.2, 3.7) | N/A | 3.1 (2.9, 3.4) |
| PANSS | 15.9 (5.3) | −1.87 (−2.8, −0.94)** | −1.79 (−2.91, −0.66)* | 16.4 (5.5) | −1.6 (−2.6, −0.64)* |
| Positive | | | | | |
| PANSS | 16.9 (6.2) | −0.67 (−1.6. 0.30) | 0.24 (−0.75, 1.24)^{d} | 15.1 (6.1) | −0.18 (−1.2, 0.82) |
| Negative | | | | | |
| PANSS | 31.6 (8.5) | −3.28 (−4.7, −1.9)** | −1.6 (−3.4, 0.06)^{d} | 32.5 (8.0) | −1.7 (−3.2, −0.26)* |
| General | | | | | |
| PANSS | 64.4 (16.3) | −6.23 (−8.8, −376)** | −2.9 (−5.8, 0.90)^{d} | 64.0 (15.4) | −3.6 (−6.3, −0.90)* |
| Total | | | | | |
| GAF | 49.3 (12.8) | 2.2 (−0.18, 4.6) | 1.9 (−1.02, 4.72) | 50.6 (15.1) | 2.7 (0.22, 5.3)* |
| SOFAS | 50.9 (9.9) | −0.45 (−3.2, 2.3) | −1.6 (−5.18, 1.96) | 56.6 (12.4) | −0.25 (−3.5, 3.0) |
| BAS | 0.86 (1.5) | −0.03 (−0.37, 0.30) | 0.12 (−0.21, 0.46) | 0.96 (1.8) | −0.23 (−0.58, 0.11) |
| SAS | 1.4 (1.7) | −0.11 (−0.35, 0.13) | −0.05 (−0.33, 0.22) | 1.9 (1.6) | −0.05 (−0.30, 0.21) |
| AIMS | 1.7 (3.0) | −0.23 (−0.77, 0.31) | −0.32 (−0.87, 0.24) | 2.7 (4.6) | 0.08 (−0.48, 0.65) |

| | | Between Placebo-NAC differences | |
|---|---|---|---|
| | | | Week 24 |
| | Within NAC Group | Week 8 | LS Mean |
| | Week 24 | LS Mean Difference | Difference |
| Outcome | Mean Overall^{b} Change | (95% CI) | (95% CI) |
| Measure | (95% CI) | p-value^{a} | p-value^{a} |
| CGI-S | −0.35 (−0.56, −0.14)* | 0.24 (0.03, 0.45)* | 0.32 (0.05, 0.59)* |
| CGI-I^{c} | 2.9 (2.6, 3.1) | N/A | N/A |
| PANSS | −2.3 (−3.49, −1.09)** | −0.25 (−1.5, 0.98) | 0.50 (−1.1, 2.1) |
| Positive | | | |
| PANSS | −1.6 (−2.71, −0.46)^{d} | −0.49 (−1.8, 0.80) | 1.8 (0.32, 3.3)^{d}* |
| Negative | | | |
| PANSS | −4.4 (−6.39, −2.48)^{d}** | −1.5 (−3.4, 0.34) | 2.8 (0.20, 5.4)^{d}* |
| General | | | |
| PANSS | −8.8 (−12.2, −5.5)^{d}** | −2.6 (−6.1, 0.80) | 6.0 (1.5, 10.4)^{d}* |
| Total | | | |
| GAF | 4.5 (1.5, 7.5)* | −0.51 (−3.7, 2.7) | −2.6 (−6.6, 1.3) |
| SOFAS | −0.69 (−4.99, 3.6) | −0.20 (−3.9, 3.5) | −0.92 (−5.67, 3.82) |
| BAS | −0.42 (−0.77, −0.06)* | 0.20 (−0.24, 0.64) | 0.54 (0.08, 1.00)* |
| SAS | −0.17 (−0.46, 0.13) | −0.06 (−0.39, 0.26) | 0.11 (−0.27, 0.50) |
| AIMS | −0.44 (−1.03, 0.16) | −0.31 (−1.0, 0.41) | 0.12 (−0.65, 0.89) |

Abbreviations:
LS Mean, Least Squares Mean;
CI, confidence interval.
^{a}Between treatment group LSmeans, CI and p-values are from LOCF ANCOVA model with terms baseline score, treatment and investigator.
^{b}Within treatment group LSmeans, CI and p-values are from LOCF ANCOVA model with terms baseline score, treatment and investigator.
^{c}CGI-I does not measure baseline score. All subsequent measures refer to baseline status. Mean (CI) refers to score at that time point
^{d}Within and between treatment group LSmeans, CI and p-values are from LOCF ANCOVA model with terms baseline score, treatment, investigator and treatment by investigator (interaction).
Population: All randomised patients
*mean difference significant at 0.05
**mean difference significant at 0.001

TABLE 4
Primary and Secondary Outcome Measures at Week 24 and Change at Post-treatment Discontinuation (washout, week 28)

| | Placebo | | NAC | | LS Mean Difference |
|---|---|---|---|---|---|
| | Mean | Mean Overall^{b} Change | Mean | Mean Overall^{b} Change | (95% CI) |
| Outcome Measure | Week 24 (SD) | (95% CI) | Week 24 (SD) | (95% CI) | p-value^{a} |
| CGI-S | 4.0 (1.1) | −0.06 (−0.30, 0.18) | 3.5 (1.0) | −0.17 (−0.41, 0.08) | 0.10 (−0.23, 0.44)^{‡} |
| CGI-I^{c} | 3.5 (1.1) | 3.5 (3.0, 3.9) | 2.9 (0.93) | 2.9 (2.5, 3.4) | N/A |
| PANSS Positive | 14.2 (5.9) | −0.21 (−1.3, 0.84) | 14.5 (5.6) | −0.96 (−2.0, 0.12) | 0.75 (−0.72, 2.22) |
| PANSS Negative^{d} | 15.9 (6.5) | −0.98 (−2.1, 0.10) | 13.7 (4.9) | 0.60 (−0.55, 1.7) | −1.58 (−3.17, 0.00)^{‡} |
| PANSS General^{d} | 29.1 (10.0) | −0.68 (−2.5, 1.16) | 28.8 (8.5) | 0.18 (−1.7, 2.1) | −0.85 (−3.45, 1.75)^{‡} |
| PANSS Total^{d} | 59.2 (19.3) | −2.1 (−5.0, 0.68) | 57.0 (16.1) | 0.56 (−2.4, 3.6) | −2.70 (−6.85, 1.45)^{‡} |
| GAF | 49.8 (14.8) | 0.98 (−1.9, 3.86) | 54.4 (15.4) | 0.37 (−2.7, 3.5) | 0.61 (−3.5, 4.72)^{‡} |
| SOFAS | 51.2 (12.8) | −2.0 (−4.8, 0.91) | 58.8 (12.7) | −0.79 (−3.8, 2.2) | −1.17 (−4.89, 2.55) |
| BAS | 0.86 (1.7) | 0.11 (−0.31, 0.53) | 0.42 (0.93) | 0.42 (−0.02, 0.86) | −0.31 (−0.91, 0.29)^{‡} |
| SAS | 1.2 (1.6) | −0.22 (−0.56, 0.11) | 1.6 (1.3) | 0.30 (−0.06, 0.65) | −0.52 (−0.99, −0.05)* |
| AIMS | 1.2 (2.9) | −0.67 (−1.3, −0.02)* | 2.2 (3.5) | −0.27 (−1.0, 0.50) | −0.40 (−1.41, 0.61) |

Abbreviations:
LS Mean, Least Squares Mean;
CI, confidence interval.
^{a}Between treatment group LSmeans, CI and p-values are from LOCF ANCOVA model with terms baseline score, treatment and investigator.
^{b}Within treatment group LSmeans, CI and p-values are from LOCF ANCOVA model with terms baseline score, treatment and investigator.
^{c}CGI-I does not measure baseline score. All subsequent measures refer to baseline status. Mean (CI) refers to score at that time point
^{d}Within and between treatment group LSmeans, CI and p-values are from LOCF ANCOVA model with terms baseline score, treatment, investigator and treatment by investigator (interaction).
^{‡}Significant improvement at week 24 that was not evident after post-treatment discontinuation (washout, week 28).
Population: All randomised patients
*mean difference significant at 0.05
**mean difference significant at 0.001

### Results

### Study Population

A total of 665 people were screened to take part in the trial. Of these, 525 people were not enrolled and 140 were enrolled, of which 71 were randomised into the placebo group and 69 were randomised into the treatment (NAC) group. A total of 111 participants completed the acute phase (up to week 8), 84 completed the maintenance phase of the trial (week 24) and 61 completed the post-discontinuation visit. The most common reason for non-completion in this sample was the withdrawal of consent by participants. Table 1 shows the disposition flowchart.

There was no significant difference between the two groups for any of the baseline measures (Table 2). The mean age of the sample was 36.6 years, and there were 42 females and 98 males. The average duration of illness was 12.2 years with participants reporting a mean of 2.1 admissions (median 1) over the course of their illness. Comorbid psychiatric diagnoses were overall similar in both groups but there were a significantly higher numbers of individuals with social phobia (N=22) and substance abuse (N=13) in the placebo group compared with the NAC group (N=11 and 4, respectively). Any suicidal ideation on the MINI was endorsed by 54% of respondents. Of the participants that responded (N=138), 4 participants in the placebo group, and 5 people in the NAC group indicated that they had had a previous suicide attempt. A positive family history of schizophrenia was seen in 17%, depression in 31%, anxiety in 8% and bipolar disorder in 7%, but there were no treatment group differences.

Clozapine (45% of participants) and olanzapine (20% of participants) were the two most commonly used primary antipsychotics. There was no significant difference between the treatment groups in this regard. Other atypical antipsychotics (risperidone, quetiapine and aripiprazole) and typical depot antipsychotics accounted for the remainder. The mean doses of chlorpromazine equivalents in the placebo group [598.2 mg (SE 56.1)] and the NAC group [716.4 mg (SE 57.0)] were not significantly different. There was a non-significant mean dose increase of 20.6 mg chlorpromazine equivalents in the NAC group and 73.1 mg in the placebo group between visits 1 (baseline) and 9 (week 24). Similarly, other medications, broken down into antidepressants, benzodiazepines, antipsychotics (other than primary) and ‘other’ were recorded at baseline. The sample did not significantly differ between groups on this parameter. Treatment adherence data was determined by an audit of returned medication packs, which found a non-significant 5.9% and 2.2% discrepancy in the placebo and NAC groups respectively over the 24 week treatment period.

Kaplan-Meier survival analysis showed that the dropout rate over the 28-week trial period for all reasons, for patient-initiated reasons (withdrew consent, lost to follow up, non-adherent, non-compliant or non-reliable), or for clinician-initiated reasons (adverse event, added mood stabilizer, primary antipsychotic changed or stopped, withdrawal by investigator) was not different between the NAC and placebo groups (p>0.1 for all comparisons).

### Primary Outcome Measures

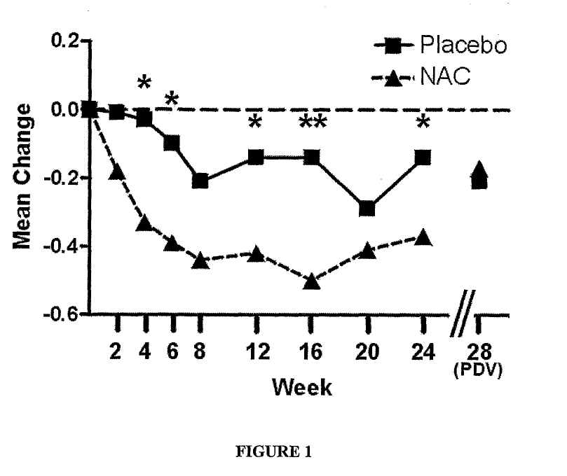

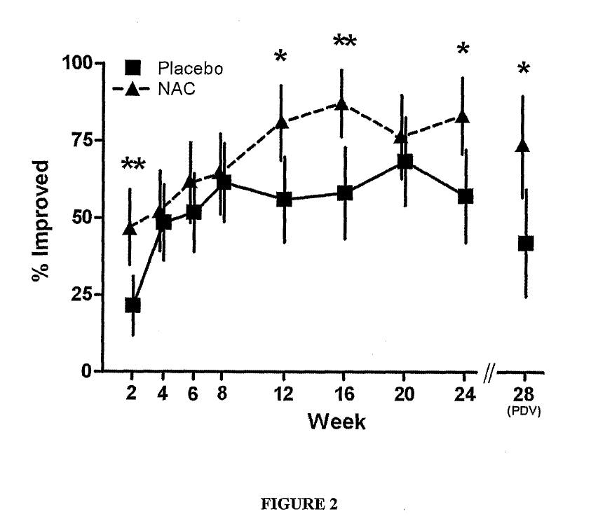

CGI-S scores on average, reduced significantly over all visits for the NAC treatment group compared to the placebo group (mean difference [95% CI]: −0.26 [−0.08, −0.44], p=0.004; Table 3, FIG. 1). Similarly, for CGI-I scores, NAC-treated subjects exhibited a greater clinical improvement than placebo-treated controls over all visits (mean difference [95% CI]: −0.22 [−0.03, −0.41], p=0.025, Table 3, FIG. 2).

The onset of clinical benefit was rapid on the CGI scales, with scores significantly better (using MMRM analysis for CGI-S, and Fisher's categorical analysis for CGI-1) in the NAC treatment group compared to the placebo group within 2 weeks (CGI-S, FIG. 1) and 4 weeks (CGI-I, FIG. 2) of commencing treatment. LOCF analyses of CGI-S scores at only two intervals: at the end of the acute treatment phase (Week 8) and at the end of the maintenance treatment phase (Week 24) was performed. This confirmed that NAC treatment induced improvement compared to placebo at both intervals (Week 8, p=0.027; Week 24, p=0.022; Table 3).

While the placebo group improved between weeks 4-8, so that significance of the difference between groups was lost on CGI-I in that interval and on CGI-S at week 8, overall the benefit of NAC treatment compared to placebo was sustained over the treatment interval (24 weeks) with significant improvement at weeks 4, 6, 12, 16 and 24 on CGI-S (FIG. 2). At weeks 12, 16 and 24 significantly more subjects (≈25%) in the NAC treatment group showed improvement on CGI-I compared to placebo (FIG. 1). We also performed ANCOVA for CGI-S scores at two predefined intervals. Table 3 shows illustrative data from the end of week 8 (a customary treatment interval for antipsychotic trials) and from the end of treatment (week 24). NAC-treated subjects improved compared to placebo at both intervals (Week 8, LS Mean Difference 0.24, p=0.027; Week 24, LS Mean Difference 0.32, p=0.022; Table 3). To clarify the magnitude of the differential clinical improvement between NAC and placebo groups in mean CGI-S scores, we also analyzed the shifts in CGI-S scores from baseline. MMRM analysis revealed that the maximum difference between placebo and NAC groups was at 16 weeks of treatment (FIG. 1). At that visit, 9 out of 44 remaining placebo subjects had improved by 1 or more CGI-S points (range 1-2) from their baseline scores. By comparison, NAC treatment was associated with 21 out 44 remaining subjects improving from baseline (p=0.007), by a range of 1-3 points. Therefore, while the differences in CGI-S scores between NAC and placebo groups were small when expressed as averages, the number of patients who exhibited a clinician-observed improvement was more than two-fold greater in the NAC group than in the placebo group.

To characterize the quality of the clinical improvement detected by the CGI-I, a MMRM analysis on improvers for all outcomes was performed. The improvement on CGI-I was found to be significantly accompanied by improvement on PANSS positive, negative, general and total subscales, as well as on CGI-S, GAF and SOFAS, but not on the SAS, BAS or AIMS. Therefore, the treatment effect observed on CGI-I probably reflects improvement of schizophrenia symptoms and not merely general health.

There were significantly greater improvements observed in the NAC treatment group compared to the placebo group for PANSS Negative (LS Mean Difference 1.8, p=0.018), PANSS General (LS Mean Difference 2.8, p=0.035) and PANSS Total (LS Mean Difference 6.0, p=0.009) scores at Week 24 when compared to baseline using ANCOVA (Table 3). However, there were no differences observed in PANSS measures when comparing changes from baseline to week 8 (Table 3), suggesting that the clinical benefit was dependent on duration of exposure to NAC. We also performed MMRM analysis on the PANSS scales, but did not detect a significant difference between NAC and placebo over all visits. However, MMRM analysis of individual items on the PANSS scales did reveal a significant (p<0.05) benefit of NAC on items 3 and 6 on the PANSS-Negative over all visits (p=0.0509 for PANSS-General item 16), as well as significant (p<0.05) improvements on several items on the PANSS scales at specific visits: at week 2 PANSS-Positive item 6, at week 4 PANSS-General item 16, at week 8 PANSS-Negative item 3, at week 16 PANSS-Negative items 3 and 7, and PANSS-General item 16, at week 20 PANSS-Positive item 7, PANSS-Negative items 3 and 7 and PANSS-General item 11, and at week 24 PANSS-Positive item 2, PANSS-Negative item 3 and PANSS-Negative item 6. The occasions when NAC treatment was significantly better than placebo on a PANSS item clearly increased in frequency as the trial progressed (6 instances in the first 7 visits, and 7 instances in the last 2 visits on treatment). In contrast, placebo treatment was significantly better than NAC on only one occasion, at week 8, on PANSS-General item 12 (data not shown). A caveat in this sub-analysis is that the type 1 error rate may be exaggerated, however given the exploratory nature of the study we felt it important to gauge the signals achieved on the PANSS in depth.

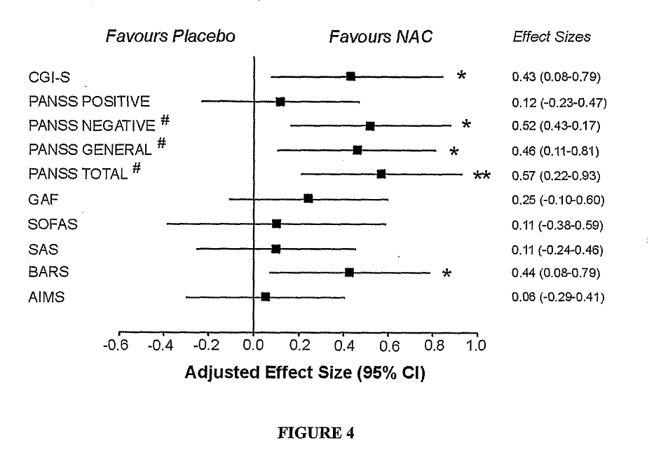

There were no between-group differences on functioning, as measured by the GAF scale or the SOFAS (FIG. 4). However, ANCOVA revealed a significant within-group improvement from baseline to endpoint on the GAF scale (mean overall change of +4.5 points) for the NAC treatment group but not for the placebo group (mean overall change of +1.9 points, Table 3). This was also confirmed by MMRM analysis where the average improvement over all visits of the NAC group was significant (+3.1 points, p=0.0026), but the overall average change from baseline (+1.5 points) for the placebo group was not significant.

Post-hoc analyses revealed no differences between NAC and placebo groups for baseline predictors of outcome: treatment (clozapine compared to other antipsychotics) gender, age, duration of illness, comorbidity and number of hospitalizations.

A calculation of the effect sizes (Cohen's d) of the benefits after 24 weeks of NAC treatment on CGI-S, PANSS Negative, PANSS General, and PANSS Total rating scales, revealed moderate improvements ranging from 0.43 to 0.57 (FIG. 4).

There were no differences between NAC and placebo groups in scores on the Global Assessment of Functioning Scale (GAF) or the Social and Occupational Functioning Scale (SOFAS, FIG. 4).

### Post Discontinuation Measures

The treatment benefit of NAC on CGI-S at the treatment phase endpoint (Week 24) was lost upon washout (Week 28, the post discontinuation visit) (mean difference [95% CI]: −0.10 [−0.23, 0.44], p=0.54; Table 4, FIG. 1). However, the proportion of patients who were clinically improved when referred to baseline, on the CGI-I scale, remained significantly greater in the NAC group at week 28 (FIG. 2). Similarly, the significant improvement for the NAC group observed at week 24 on scores for PANSS Positive, PANSS General, PANSS Total and BAS, were not evident after treatment discontinuation (Table 4). In addition, the significant within NAC group improvement on GAF scores at week 24 was lost post-discontinuation (Table 4).

### Effects on Abnormal Movements

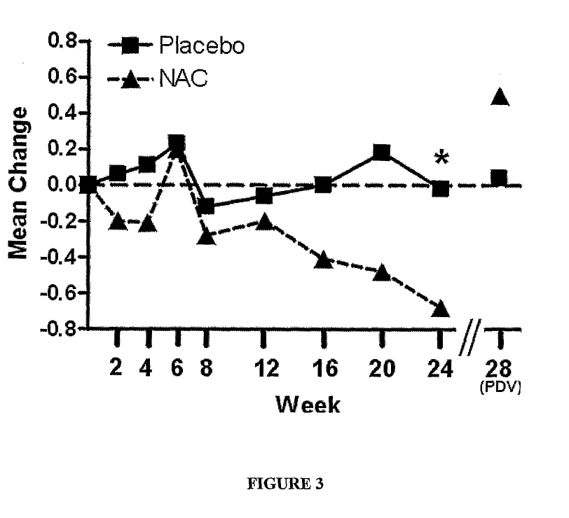

Over all visits, there were no significant differences detected between the placebo and NAC groups on the SAS or AIMS scores. Baseline to week 24 LOCF endpoint changes indicated that the NAC group had improved akathisia on the BAS scale compared the placebo group as a product of time on treatment (FIG. 3), with the difference between treatment groups reaching significance at week 24 (p=0.022). A calculation of the effect size (Cohen's d statistic) of the benefits after 24 weeks of NAC treatment on the BAS revealed a low-medium improvement of 0.44 (FIG. 4).

### Adverse Effects and Safety

Overall, there were no significant effects of NAC on any safety parameters, including vital signs, weight and clinical biochemistry values. Adverse events were recorded based on participant reports throughout the trial using a checklist of 44 somatic items. No reported event was significantly more common in the NAC group compared to placebo group, except for significantly less eye irritation in the NAC treatment group (p=0.034).

Covarying for baseline revealed no significant differences in change in weight between groups at week 8 or week 24. Mean weight gain at week 8 for the placebo group was 1.748 kg (SE 0.575), n=46 and 1.372 kg (SE 0.517), n=43 for the NAC group. At week 24 the mean gain in weight in the placebo was 1.159 kg (SE 0.874), n=27 and in the NAC group was 0.394 kg (SE 0.932), n=33. There were three serious adverse events recorded during the course of the trial all of which were hospital admissions for non-adherence to primary antipsychotics, and all occurred in the placebo group.

### Discussion

The results of this study indicate that adjunctive treatment of chronic schizophrenia with 2 g/day oral NAC reduces clinical severity as measured by PANSS and CGI-S scores, and improves global measures of symptomatology as measured by CGI-I scores (FIG. 2), with a clinical effect size comparable to initiating clozapine treatment (Pickar D. and Bartko J., 2003) (FIG. 4). While both trial groups were treated with standard antipsychotic medication, ≈25% more participants taking adjunctive NAC demonstrated clinical improvement on the CGI-1 than participants on placebo at weeks 12, 16 and 24 (FIG. 2). On the PANSS, there was a significant treatment by investigator interaction. Controlling for this, the results for PANSS negative, total and general were significant in favour of the NAC group. It is recommended in the literature that investigator by treatment interactions are controlled for, and for this reason the interaction term was left in the model.

A significant moderate benefit of NAC at endpoint for akathisia was also evident (FIG. 3) on the BAS. The lack of effect on the AIMS and SAS may reflect the very low basal scores in these measures, given that atypical antipsychotic medications, particularly clozapine were the predominant maintenance medication in the study cohorts.

At the maximum point of differentiation between NAC and placebo groups, the raters detected clinical improvement from baseline (using CGI-S) in more than twice as many NAC-treated subjects compared to placebo-treated subjects (p=0.007). Further supporting the likelihood of a NAC treatment effect, several of the significant benefits that were detected were lost after a 4-week washout (FIG. 1, Table 2).

While the improvement on CGI scales was detected by the more stringent MMRM analysis, improvements on the PANSS Negative, Total and General scales were observed using ANCOVA LOCF, which does not fully account for treatment effects on the dropouts. However, survival analysis found no significant difference in the dropout rates between NAC and placebo groups for either clinician- or patient-initiated reasons. In addition, the majority of withdrawals from the study could be explained by the data observed, only three patients were lost to follow up, and discontinuation rates were similar between the groups, supporting the likelihood that clinical data on the dropouts are missing at random and therefore the MMRM analysis is valid.

The MMRM analysis also found that improvement on the CGI scale was accompanied by significant improvement on the PANSS subscales, suggesting that the observed clinical improvement was likely driven by resolution of psychotic illness. Also, significant improvements that became more frequent at later visits were identified by MMRM on eight PANSS sub-items.

Taken together these findings indicate that NAC treatment improves schizophrenia symptoms. As the first randomized clinical trial of its kind, this study has probed clinical parameters without being able to be powered for a primary outcome but has nevertheless revealed significant improvement on several outcomes, suggestive of a real clinical benefit. While the PANSS outcomes reached significance on the ANCOVA, they did not reach significance on the more stringent MMRM analysis, which may be due to underpowering.

A significant moderate benefit of NAC at endpoint for akathisia was also evident (FIG. 3) on the BAS. The lack of effect on the AIMS and SAS may reflect the low basal scores in these measures, because of atypical antipsychotics being the predominant maintenance medication. These results support further examination of NAC as a neuroprotective treatment for extra pyramidal symptoms (EPS).

### Example 2

### N-acetyl Cysteine in Bipolar Disorder: A Double Blind Randomised Placebo Controlled Trial

There is evidence of oxidative stress in bipolar disorder. Glutathione is a key endogenous free radical scavenger, and N-acetyl cysteine (NAC) is a well-tolerated, orally-bioavailable precursor of glutathione.

### Methods

### Study Design

Consented individuals were assigned using simple randomization (Beller et al., 2002) to treatment with NAC or placebo in addition to treatment as usual, in a double-blind fashion. The nature and dose of the primary therapy was monitored. The person generating the randomisation schedule was not involved in any aspect of participant interview. The investigators, clinicians and statisticians were blind to treatment allocation until the data analysis was completed. The study was registered with the Australian Clinical Trials Registry (Protocol #12605000362695). Participants were recruited through advertisements, their private psychiatrists and database screening. All participants provided written informed consent. The trial setting was in the public and private outpatient psychiatry clinics of the participating centers. The trial was approved by each participating research and ethics committee (Barwon Health, Southwest Area Mental Health Service, Bendigo Health, all in Victoria, Australia), and was conducted according to Good Clinical Practice guidelines.

This was, to the inventors' knowledge, the first clinical trial of NAC for bipolar disorder. Therefore, there was no prior data to power our exploratory study for a primary readout. The study was powered to detect moderate effect sizes of 0.5 to 0.8 in trials of psychotropic medication (Cohen, 1988).

NAC was acquired from Zambon, Italy. Purity was 99.8% as determined by HPLC. DFC Thompson, Sydney, Australia, performed encapsulation of both the NAC and the placebo capsules. Bottles were sealed, dispensed by pharmacy, and returned to pharmacy so that the investigators were not exposed to the contents of the bottles. Participants were seen separately, and had no opportunity to compare experiences. Pill counts for adherence were done by the pharmacy, and an independent person confirmed the capsule audit.

### Dose Rationale

All randomized participants received two NAC (500 mg) capsules twice daily (2 g daily), or matching placebo. We selected a daily dose that was at the upper dosing range for published clinical trials of oral NAC of 12 weeks to 12 months duration, and that reported evidence of tolerability and some efficacy (Adair et al., 2001; Van Schooten et al., 2002; Behr et al., 2002; Demedts et al., 2005).

### Inclusion and Exclusion Criteria

Individuals needed to meet DSM-IV criteria for bipolar disorder (I or II) with at least 1 documented episode of illness (depressive, manic or mixed) in the previous 6 months, and had to have been on stable therapy for at least 1 month prior to randomisation. Participants were required to have the capacity to consent to the study and comply with study procedures, and utilise effective contraception where indicated.

Exclusion criteria included individuals with a known or suspected clinically relevant systemic medical disorder, including asthma, bronchospasm, or respiratory insufficiency, recent gastrointestinal ulcers, and individuals who were pregnant or lactating. Individuals taking greater than 500 mg of NAC/day, 200 μg of selenium/day or 500 IU of Vitamin E/day were excluded, as were those with a history of anaphylaxis with NAC or any component of the preparation. Inability to comply with either the requirements of informed consent or the treatment protocol was also an exclusion criterion.

Withdrawal from the study occurred if participants ceased taking their trial medication for 7 consecutive days, stopped effective contraception or became pregnant. Dose changes to existing medications, or the addition or removal of an agent were accepted, and participants were allowed to continue with the study. Participants were withdrawn from the study if they revoked their consent or developed serious adverse events associated with the study drug, which could occur either at the request of the patient or the discretion of the investigator.

### Participant Evaluation

Participants were assessed at baseline using a structured clinical interview (MINI-plus) and underwent a physical examination. Clinical status was assessed using the MADRS, BDRS, YMRS, CGI-Improvement and Severity scales for bipolar disorder (CGI-I-BP, CGI-S-BP), GAF, SOFAS, SLICE/LIFE, LIFE RIFT, and Q-LES-Q.

These scales were repeated 2 weekly for the first 4 weeks and 4-weekly thereafter for a total of 24 weeks, or on the day of study termination if the patient withdrew prior to the final scheduled visit. A follow-up visit was conducted 4 weeks (±2 weeks) after the trial completion, either at trial endpoint (week 24), or premature discontinuation, to determine any change in clinical status on treatment discontinuation.

Time to any intervention for mood symptoms was a further outcome measure. This was defined as initiation of a new medication, initiation of emergency medical contact, psychotherapy, hospitalisation or electroconvulsive therapy (ECT), or discontinuation or dose adjustment of a current agent, all in response to a clinician's assessment of a new mood episode. Adherence was monitored using pill counts of returned clinical trial material. Adverse events were tabulated. Serious adverse events were reported to all research and ethics committees and also to the Therapeutic Goods Administration.

Randomization occurred at Visit 1. Trial endpoint was defined as the last post-baseline value obtained for a participant for a given measure during the treatment phase. For participants who completed the protocol, this corresponded to the Visit 8 (week 24) assessment. All randomized participants who had at least one post-baseline assessment were included in the analysis.

### Statistical Analysis

Analysis was performed by an external consultant statistician, who was blind to treatment assignment, using SAS version 8.2 for Windows (SAS Institute, Cary, N.C.), on a clean and locked database. All analyses were conducted in accordance with the International Conference on Harmonization E9 statistical principles (International Conference on harmonisation: guidelines on statistical principles for clinical trials (ICH-E9) 1997) and are based on all randomized patients with at least one post-baseline observation (intention to treat population).

The efficacy analysis assessed average treatment group differences for each of the outcomes measured over the entire study period, and used a likelihood based mixed-effects model, repeated measures approach (MMRM). The MMRM model included the fixed, categorical effects of treatment, investigator, visit, and treatment-by-visit interaction, as well as the continuous, fixed covariates of baseline score and baseline score-by-visit interaction. The MMRM includes all available data at each time point (Mallinckrodt et al., 2004). In addition, Kaplan Meier estimates and the log-rank test were used to evaluate time to a mood episode.

Results from the analysis of dichotomous data are presented as proportions, with 95% confidence interval, and Fisher's Exact p-value where appropriate.

Effect sizes were calculated at endpoint using MMRM. Applying Cohen's guidelines (Cohen, 1998), an effect size of 0.2-0.4 is considered a small effect, 0.5-0.7 is considered a medium effect and ≧0.8 is considered a large effect. For all other secondary measures (quality of life and functioning), the above analysis was utilised as described.

All tests of treatment effects were conducted using a two-sided alpha level of 0.05 and 95% confidence intervals were presented. The term significant in this report indicates statistical significance (P≦0.05).

### Results

### Study Population

One hundred and eighty three people were screened to take part in the trial. Of these, 108 people were not eligible and 75 were eligible and enrolled, of which 37 were randomised into the placebo group and 38 were randomised into the treatment (NAC) group. Forty-eight participants completed the full 24-week trial period and 58 (including individuals who terminated prematurely) completed the post-discontinuation visit (Table 5). The most common reason for non-completion in the trial was withdrawal of consent by participants. In the NAC group 31/38 (81.58%) of participants had a diagnosis of bipolar I disorder, and 30/37 (81.08%) in the placebo group. In the NAC group, 7/38 (18.42%) of participants had a diagnosis of bipolar II disorder, and 7/37 (18.92%) in the placebo group. There were no significant differences between the NAC and placebo groups in this regard. The two groups were also matched on the baseline demographic and clinical measures (Table 6). The mean age of the sample was 45.6 years, and there were 45 females and 30 males. The average duration of illness was 10.25 years with participants reporting a mean of 2.35 admissions (median 1) over the course of their illness. There were no differences in comorbid psychiatric diagnoses between the groups.

TABLE 5

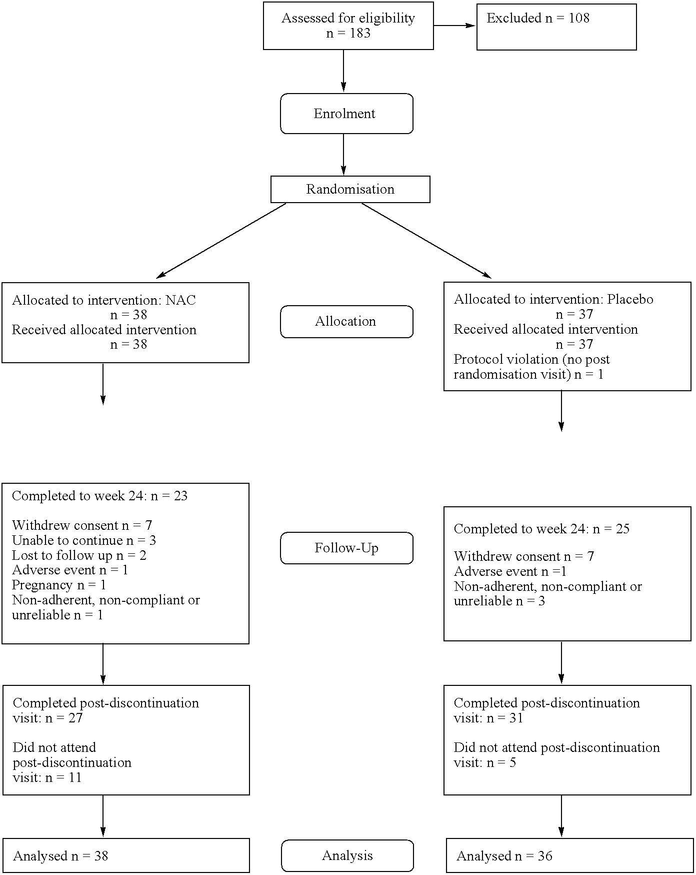

TABLE 6
Patient Characteristics

| | NAC | Placebo | Overall |
|---|---|---|---|
| | (N = 38) | (N = 37) | (N = 75) |
| Measures | n (%) | n (%) | n (%) |
| Age (years)* | 44.6 (11.2) | 46.6 (13.8) | 45.6 (12.5) |
| Male Gender | 15 (39.5) | 15 (40.5) | 30 (40) |
| Public Treating Sector | 10 (26.3) | 8 (21.6) | 18 (24) |
| Medication (Baseline) | | | |
| Valproate | 15.0 (39.5) | 16 (43.2) | 31 (41.3) |
| Lithium | 15 (39.5) | 13 (35.1) | 28 (37.3) |
| Carbamazepine | 1 (2.6) | 2 (5.4) | 3 (4.0) |
| Lamotrigine | 5 (13.2) | 1 (2.7) | 6 (8.0) |
| Antipsychotics | 15 (39.5) | 18 (48.7) | 33 (44.0) |
| Antidepressants | 17 (44.7) | 19 (51.4) | 36 (48.0) |
| Benzodiazepines | 8 (21.1) | 8 (21.6) | 16 (21.3) |
| Others | 1 (2.6) | 1 (2.7) | 2 (2.7) |

| | NAC | | Placebo | | |
|---|---|---|---|---|---|
| NUMBER OF EPISODES** | (Median, Range) | (Min, Max) | (Median, Range) | (Min, Max) | P-value |
| Manic | 6.5, 69 | 1, 70 | 4.5, 69 | 1, 70 | 0.995 |
| Hypomanic | 8.5, 69 | 1, 70 | 4.0, 69 | 1, 70 | 0.959 |
| Depressive | 22.0, 73 | 1, 74 | 7.0, 99 | 1, 100 | 0.545 |
| Suicide Attempt | 2.0, 5 | 1, 6 | 2.0, 5 | 1, 6 | 0.755 |

*Data are given as mean and standard deviation.
**Median values are given

There were no statistically significant differences on any comparison between NAC and placebo groups for any of the items in this table.

### Efficacy Outcomes

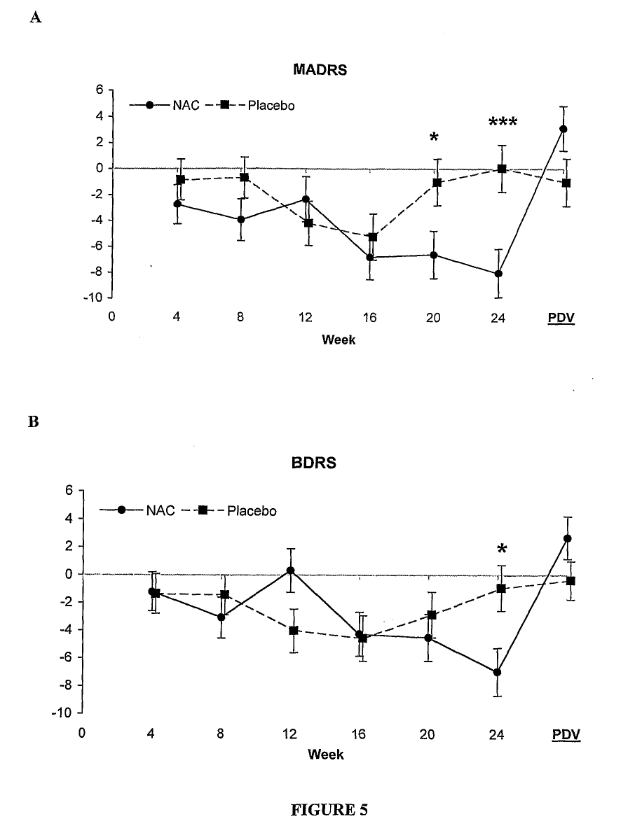

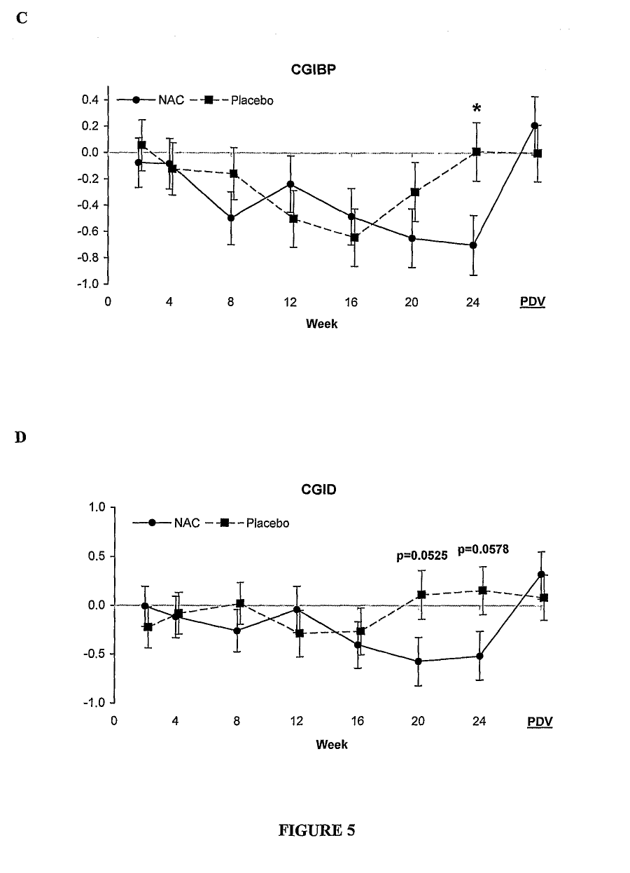

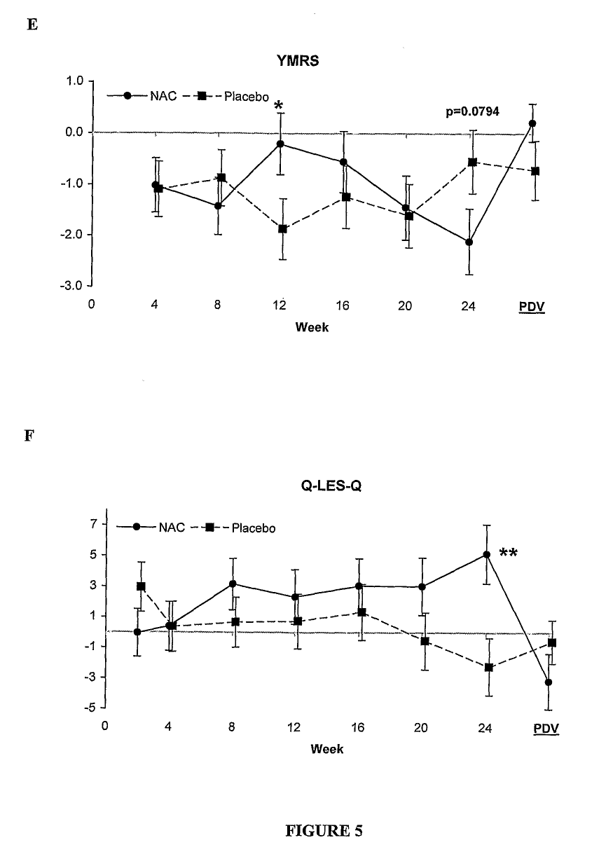

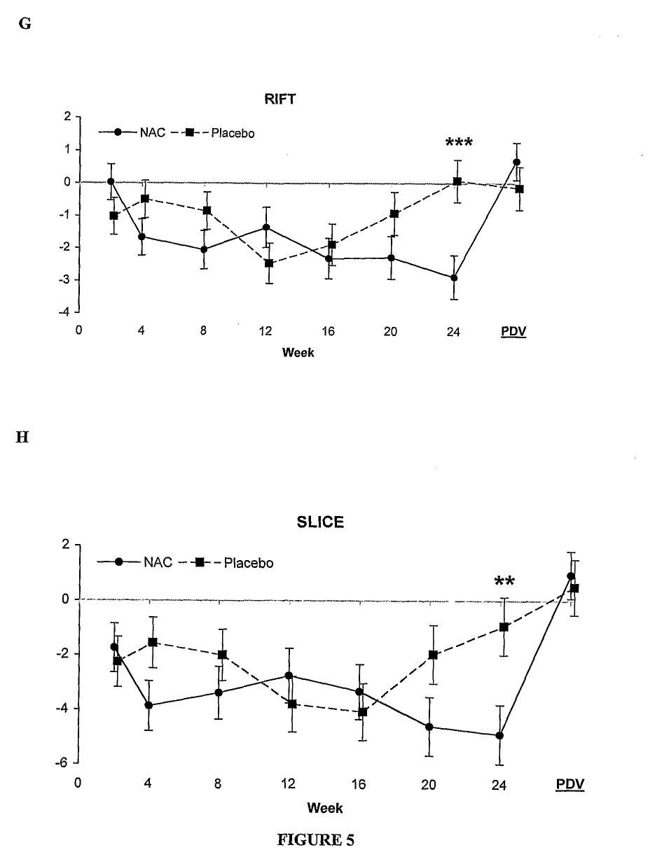

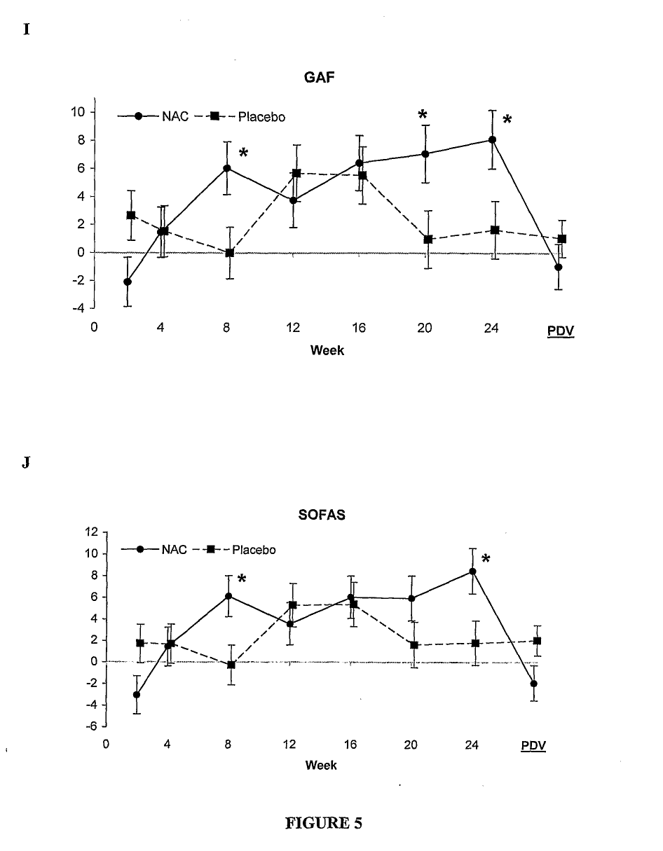

There was a significant reduction in symptoms at treatment completion (week 24) on most symptomatic measures used in the trial (Table 7). These included the MADRS (LS mean difference [95% CI]: −8.05 [−13.16, −2.95], p=0.002) (Table 7; FIG. 5A) and BDRS (LS mean difference [95% CI]: −6.01 [−10.96, −1.34], p=0.012) (Table 7, FIG. 5B). The MADRS result was also significant at the 20 week visit (LS mean difference [95% CI]: −5.57 [−10.61, −0.53], p=0.031). There was a significant advantage of NAC over placebo measured by the CGI-S-BP (LS mean difference [95% CI]: −0.71 [−1.33, −0.09], p=0.026) (FIG. 5C, Table 7). On scores of CGID, there was a non-significant trend (FIG. 5D). On scores of mania, there was a non-significant trend in favour of NAC treatment evident on the YMRS (LS mean difference [95% CI]: −1.56 [−3.31, −0.18], p=0.079) (Table 7, FIG. 5E). Baseline mania scores however were low (NAC mean baseline [95% CI]: 4.08 [2.72, 5.44], placebo mean baseline score [95% CI]: 4.03 [2.52, 5.53]).

TABLE 7
Outcome Measures at Baseline, Week 24 and Change at Post-treatment Discontinuation (washout, week 28)

| | | | End point to Week 24 | | Post-treatment to | | | | | |
|---|---|---|---|---|---|---|---|---|---|---|
| Outcome | Baseline (Mean ± SD) | | (Mean ± SD) | | Week 28 (Mean ± SD) | | NAC-Placebo at Endpoint* | | | |
| measures | NAC | Placebo | NAC | Placebo | NAC | Placebo | LS Mean | LCL | UCL | P-Value |
| MADRS | 16.6 (11.7) | 13.1 (9.3) | 6.6 (7.4) | 14.0 (11.5) | 12.2 (11.6) | 13.3 (11.5) | −8.05 | −13.16 | −2.95 | 0.002 |
| BDRS | 15.6 (11.6) | 12.3 (8.8) | 6.7 (6.4) | 12.0 (8.8) | 12.1 (10.8) | 11.9 (9.2) | −6.01 | −10.69 | −1.34 | 0.012 |
| CGI-S-BP | 3.5 (1.6) | 3.2 (1.2) | 2.5 (1.2) | 3.2 (1.5) | 3.1 (1.5) | 3.2 (1.7) | −0.71 | −1.33 | −0.09 | 0.026 |
| CGI-S-D | 3.1 (1.8) | 2.9 (1.4) | 2.3 (1.1) | 3.0 (1.5) | 3.1 (1.7) | 3.1 (1.7) | −0.67 | −1.36 | 0.02 | 0.058 |
| CGI-S-M | 1.9 (1.1) | 2.1 (1.0) | 1.9 (1.2) | 2.1 (1.15) | 1.9 (0.9) | 1.9 (1.1) | −0.03 | −0.49 | 0.44 | 0.908 |
| CGI-I-BP | n/a | n/a | 3.1 (1.6) | 3.6 (1.4) | 3.5 (1.5) | 3.7 (1.8) | −0.67 | −1.64 | 0.3 | 0.173 |
| CGI-I-D | n/a | n/a | 3.2 (1.6) | 3.9 (1.5) | 3.7 (1.7) | 3.9 (1.9) | −0.62 | −1.79 | 0.54 | 0.292 |
| CGI-I-M | n/a | n/a | 3.7 (1.1) | 3.6 (1.3) | 3.7 (1.0) | 3.8 (1.2) | −0.05 | −0.83 | 0.74 | 0.906 |
| YMRS | 4.1 (4.2) | 4.0 (4.5) | 2.1 (3.3) | 3.7 (5.4) | 2.8 (3.4) | 2.9 (3.1) | −1.56 | −3.31 | 0.18 | 0.079 |
| Q-LES-Q | 52.0 (11.7) | 54.4 (10.7) | 59.2 (12.7) | 51.9 (11.6) | 52.8 (11.4) | 51.1 (11.3) | 7.37 | 2.09 | 12.65 | 0.006 |
| LIFE-RIFE | 12.8 (4.2) | 10.9 (3.7) | 8.9 (3.3) | 11.5 (4.3) | 10.8 (4.2) | 11.2 (4.2) | −2.95 | −4.79 | −1.12 | 0.002 |
| SLICE-LIFE | 21.3 (6.9) | 17.9 (5.1) | 14.9 (5.2) | 18.1 (6.6) | 17.2 (6.0) | 8.0 (6.8) | −3.97 | −6.96 | −0.98 | 0.009 |
| GAF | 60.4 (11.0) | 66.1 (13.7) | 71.3 (13.9) | 67.4 (13.2) | 66.6 (14.7) | 67.7 (15.5) | 6.45 | 0.64 | 12.26 | 0.030 |
| SOFAS | 62.1 (13.1) | 66.7 (13.2) | 73.7 (13.4) | 68.3 (13.3) | 66.9 (16.2) | 69.2 (16.1) | 6.66 | 0.86 | 12.47 | 0.025 |

Abbreviations:
LS Mean, Least Squares Mean;
CI, confidence interval;
LCL Lower confidence level;
UCL Upper confidence level.
CGI-I does not measure baseline score. All subsequent measures refer to baseline status. Mean (CI) refers to score at that time point
*Between treatment group LS means at endpoint, CI and p-values are from MMRM
Population: All randomised patients

MADRS scores on average, reduced significantly over all visits for the NAC treatment group compared to the placebo group (LS mean difference [95% CI]: −3.08 [−5.99, −0.17], p=0.039). Response, defined as a 50% reduction in total MADRS score, at weeks 20 and 24 compared to baseline was observed in 46 and 51% of participants in the NAC group compared with 21 and 18% in the placebo group respectively (p=0.036 and p=0.001 respectively).

### Quality of Life & Functional Outcomes

These symptomatic changes were reflected on measures of quality of life, including the Q-LES-Q at week 24 (LS mean difference [95% CI]: 7.37 [2.09, 12.65], p=0.006; FIG. 5F), as well as the RIFT (LS mean difference [95% CI]: −2.95 [−4.79, −1.12], p=0.002; FIG. 5G) and SLICE/LIFE (LS mean difference [95% CI]: −3.97 [−6.96, −0.98], p=0.009; FIG. 5H) at endpoint (Table 7). There was a similar advantage for the NAC treated group in changes on functional measures, with significant improvement on the GAF at weeks 8, 20 and 24 (LS mean difference [95% CI]: 6.45 [0.64, 12.26], p=0.030; FIG. 5I) and SOFAS at weeks 8 (LS mean difference [95% CI]: 6.41 [1.18, 11.63], p=0.017) and 24 (LS mean difference [95% CI]: 6.66 [0.86, 12.47], p=0.025: FIG. 5J) (Table 7).

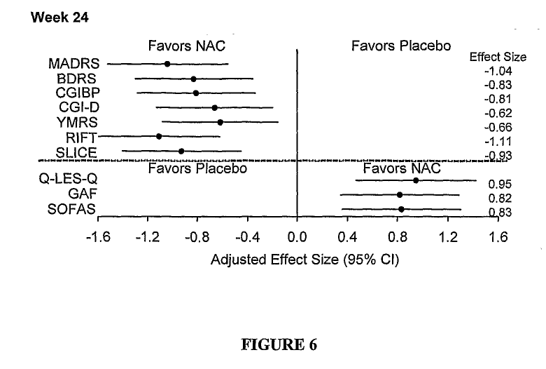

Kaplan Meier analysis did not reveal any significant differences between the two groups for time to a mood episode (Log-rank test: p=0.968). A calculation of effect sizes (Cohen's d), of the benefits of NAC treatment after 24 weeks on all rating scales, showed improvements consistent with moderate to large effects (FIG. 6).

### Post Discontinuation Measures

The treatment benefit of NAC observed at week 24, the trial endpoint was not evident at the post discontinuation visit on any of the scales included in the trial. This suggests that improvements seen in the NAC group at endpoint had been reversed by the discontinuation of NAC (FIG. 5).

### Adverse Effects

Adverse events were recorded based on participant reports throughout the trial using a checklist of 44 somatic items. Adverse events reported in more than 15% of the NAC group included changed energy (21% NAC, 27% placebo), headaches (18% NAC, 8% placebo), heartburn (16% NAC, 8% placebo) and increased pain in joints (16% NAC, 8% placebo). No reported event was significantly more common in the NAC group compared to placebo group. There were 7 serious adverse events (SAE) reported during the trial. All were hospitalisations, and all, except a victim of a motorcycle accident, were due to deteriorations in mental state. Of the 7 reported SAE's, 3 were in the NAC group and 4 were in the placebo group.

### Discussion

The results of this study suggest that adjunctive treatment of bipolar disorder with 2 g/day oral NAC causes a prominent reduction in depressive symptoms, and improvement in measures of function and quality of life over a 6-month period. There was no significant effect on symptoms of mania, although there was a trend for an effect of NAC; this may have been related to the very low baseline symptoms of mania in the cohort. It is noteworthy that the clinical benefits recorded only emerged robustly towards the end of the treatment period. As there was no uniform polarity requirement at baseline, the variance of many of the symptomatic measures was large. There was no overall effect on survival until mood episode in this study, as measured by the Kaplan Meier method, which could be consistent with the onset of action only becoming evident after several months treatment. Future studies using survival analysis could adopt an enriched design, with a run-in period on active treatment, compatible with the observed timeline of onset of action before such a survival analysis becomes meaningful. That significant differences emerged on most outcome measures with a non-enriched naturalistic sample indicates that the treatment benefit of NAC is robust. The naturalistic, multi-center, outpatient based nature of the cohort increases the generalizability of the trial.

There were no outcome differences between individuals on lithium or other mood stabilizers on post-hoc analyses, although sample sizes of the subgroups were small. Further exploration of a possible cumulative benefit with other agents is warranted, as are trials of monotherapy. There are high levels of comorbidity in bipolar disorder; it possible that these results may have been influenced by unidentified psychopathology. Furthermore, dose-finding studies are needed to determine the optimal dosing regimen of NAC for this indication.

The precise mechanism of the therapeutic benefit we observed remains to be confirmed. It is hypothesized that NAC increases brain glutathione levels, restoring the oxidative imbalances that are perturbed in bipolar disorder. However, without a direct measure of glutathione levels in the brain, as might be achieved by magnetic resonance spectroscopy (Do et al., 2000), the status of brain glutathione is uncertain.

Over time, the bulk of the burden of bipolar disorder is in the depressive pole. The management of depression in the maintenance phase is a vexing clinical issue. Currently available therapies, including antidepressants (Sachs et al., 2007), are of limited efficacy (Belmaker, 2007). The beneficial effects of NAC on depressive symptoms are of particular salience given the profile of the illness.

NAC is relatively inexpensive, available over-the-counter, and has shown safety and benefit in two randomized controlled trials for major psychiatric illness at 2 g per day for 6 months, facilitating its deployment into clinical practice. The benefits we observed indicate that disturbances in oxidative biology may play a role in bipolar disorder, and that augmentation of glutathione using NAC supplementation reduces clinical symptoms, particularly of depression, and improves functioning and quality of life in this condition over a 6 month period.

### Example 3

### Effects of N-acetyl cysteine amide (NACA) on glutathione in Rats

### Materials and Methods

### Animals

Male Sprague-Dawley rats aged nine weeks and weighing an average of 390 g, were used in the study. Animals were maintained on a twelve hour light-dark cycle with water and food ad libitum. Two animals were housed per cage with room temperature maintained at 22° C.

### Drug Administration

All pharmacological agents were administered by intraperitoneal injection (i.p.) in a volume of 1 ml/kg, except where otherwise stated. All agents were prepared using saline as a diluent. Brain GSH depletion was performed using cyclohexene-1-one (CHX), administered at 75 mg/kg. Animals were returned to their cage for 90 minutes prior to administration of N-acetyl cysteine (NAC) or N-acetyl cysteine amide (NACA or “AD4”). Animals were killed by decapitation 1 hour after NACA or NAC treatment.

### Tissue Preparation

Frontal cortex, striatum and liver samples were immediately excised on ice, weighed and sonicated in SSA buffer (5% sulfosalicylic acid in 100 mM disodium hydrogen orthophosphate, 100 mM sodium dihydrogen orthophosphate and 1 mM EDTA, pH 7.5, 5 mL/g wet tissue). The samples were subsequently centrifuged (22×10³ g for 10 minutes, 4° C.) and the homogenate was taken and frozen at −80° C. until analysed.

### Glutathione Determination

Total glutathione levels (μmol GSH/gram tissue) were determined in all samples. Samples were assayed according to Baker et al. (1990). Reduced glutathione standards ranging from 0 to 320 ρmol of GSH in 50 μL were prepared in a background buffer identical to the samples (SSA buffer) and then treated in parallel with samples. 50 μl of sample or standard was placed on a 96-well microtiter plate with 100 μL of assay reagent (final concentration in well; 0.15 mM DTNB, 0.2 mM NADPH, 1.0 U glutathione reductase/mL). Plates were assayed for a total of 2 minutes at 414 nm using a Multiskan MCC/340 MK II plate reader and Genesis V3.05 computer software. All chemicals were purchased from Sigma-Aldrich. Statistical evaluation of the data was conducted using analysis of variance (ANOVA).

### Results

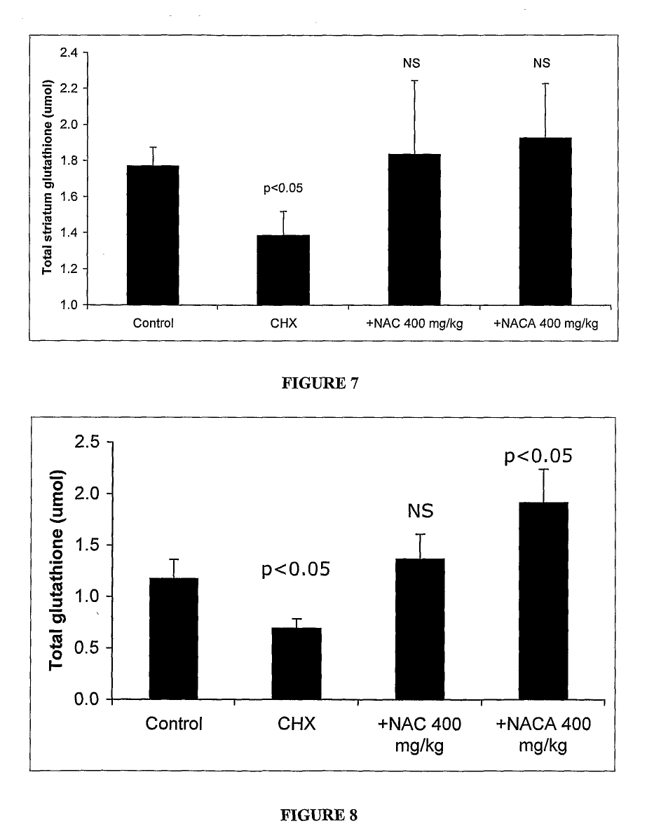

CHX treatment induced a significant decrease in total glutathione levels in the brain striatum and in the liver. This was rescued by both NAC and NACA at 400 mg/kg. In addition, NACA significantly increased liver glutathione levels above control baseline levels. (FIGS. 7 and 8).

### BIBLIOGRAPHY

Abdalla D S, Monteiro H P, Oliveira J A and Bechara E J., (1986), Clin Chem, 32:805-7
Adair J C, Knoefel J E, Morgan N., (2001), Neurology, 57:1515-7
Anil Yagcioglu A, Kivirick Akdede B, Turgut T, et al., (2005), J Clin Psychiatry, 66:63-72
Association AP. Diagnostic and statistical manual of mental disorders. 4 ed. Washington, D.C.; 1994
Baker, M. A.; Cerniglia, G. J. and Zaman, A.; Microtiter plate assay for the measurement of glutathione and glutathione disulfide in large numbers of biological samples. Anal Biochem. 190:360-365; 1990
Behr J, Degenkolb B, Krombach F, Vogelmeier C., (2002), Eur Respir J, 19(5):906-11
Beller E M, Gebski V, Keech A C. Randomisation in clinical trials. Med. J. Aust., 20002, 177(10):565-7
Belmaker R H. Treatment of bipolar depression. (2007), N. Engl. J. Med, 356(17):1771-3
Buckman T D, Kling A S, Eiduson S, Sutphin M S and Steinberg A., (1987), Biol Psychiatry, 22:1349-56
Castagne V, Cuenod M, Do K Q., (2004), Neuroscience, 123:821-34
Cohen J. Statistical power analysis for the behavioral sciences. 2 ed. Hillsdale, N.J.: Lawrence Earlbaum Associates; 1988
Demedts M, Behr J, Buhl R. et al. High-dose acetylcysteine in idiopathic pulmonary fibrosis. (2005), N. Engl. J. Med., 353(21):2229-42
Do K Q, Lauer C J, Schreiber W, Zollinger M, Gutteck-Amsler U, Cuenod M and Holsboer F., (1995), J Neurochem, 65:2652-62
Do K Q, Trabesinger A H, Kirsten-Kruger M, Lauer C J, Dydak U, Hell D, Holsboer F, Boesiger P and Cuenod M., (2000), Euro J Neurosci, 12:3721-8
Duffield A J, Thomson C D, Hill K E and Williams S., (1999), Am J Clin Nutr, 70:896-903
Gurney M E, Cutting F B, Zhai P, Doble A, Taylor C P, Andrus P K and Hall E D., (1996), Ann Neurol, 39:147-57
Henderson D, Goff D., (1996), J Clin Psychiatry, 57:395-7
Holdiness M R., (1991) Clincal Pharmokinetics, 20(2):123-34
International conference on harmonisation: guidance on statistical principles for clinical trials (ICH-E9). 1997
Kelly G S., (1998), Alt Med Rev, 3(2):114-27
Louwerse E S, Weverling G J, Bossuyt P M M, Meyjes F E P, Vianney de Jong J M B., (1995), Arch Neurol, 52:559-64.
Mallinckrodt C, Watkin J, Molenberghs G, Carroll R., (2004), Pharmaceut Statist, 3:161-9
Pickar D, Bartko J., (2003), Am J Psychiatry, 160:1133-8
Sachs G S, Nierenberg A A, Calabrese J R et al. Effectiveness of adjunctive antidepressant treatment for bipolar depression. (2007), N Engl. J. Med, 356(17):1711-22
Stern R, Schmeidler J, Davidson M., (1997), Biol Psychiatry, 42:138-43
Taylor C, Flynn S, Altman S, Ehmann T, MacEwan G, Honer W., (2001), Schizophr Res, 48:155-8
Van Schooten F J, Besaratinia A, De Flora S, et al., (2002), Cancer Epidemiol Biomarkers Prev, 11(2):167-75
Yao J K, Reddy R D and van Kammen D P., (1999), Biol Psychiatry, 45:1512-5

1. A method of treating a psychiatric or neuropsychiatric disorder comprising administering to a mammal a combination of an antipsychotic drug and a compound that increases glutathione levels in said mammal, wherein said psychiatric or neuropsychiatric disorder is selected from schizophrenia, substance abuse, psychosis, bipolar disorder, manic depression, major depression, affective disorder, schizophreniform or schizoaffective disorders, depression, psychotic depression, drug induced psychosis, delirium, autism, nausea, vertigo, inner ear infection, chronic pain, palliative care pain, agonal agitation, alcohol withdrawal syndrome, dementia induced psychosis, mood disorders and first episode psychoses.

2. A method of reducing side effects of an antipsychotic drug comprising administering to a mammal, an antipsychotic drug in combination with a compound that increases glutathione levels in said mammal, wherein the side effect is selected from drug induced Parkinsonism, acute dystonia, tachycardia, hypotension, impotence, lethargy, akathisia, seizures, hyperprolactinemia, tardive dyskinesia, diabetes, liver toxicity, cataracts, dry eyes, dysphoria and neuroleptic malignant syndrome.

3. A method according to claim 1 wherein the compound that increases glutathione levels is a compound of formula (I): 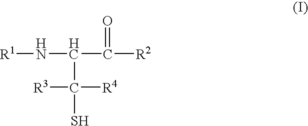
wherein
R¹ is selected from —C(O)C₁₋₄alkyl and —C(O)(CH₂)₂CH[C(O)R⁵]NHR⁶,
R² is selected from —OR⁷, —NH₂ and —NHCH₂C(O)R⁸,
R³ and R⁴ are independently selected from H and —C₁₋₄alkyl,
R⁵ is selected from —OH, —OC₁₋₄alkyl and NH₂,
R⁶ is selected from H, or C(O)C₁₋₄alkyl,
R⁷ is selected from H and C₁₋₄alkyl, and
R⁸ is selected from OH, —OC₁₋₄alkyl and NH₂,
and pharmaceutically acceptable salts thereof.

4. A method according to claim 3, wherein R¹ is selected from —C(O)CH₃, —C(O)(CH₂)₂CH(CO₂H)NHC(O)CH₃, —C(O)(CH₂)₂CH(CO₂CH₃)NHC(O)CH₃, —C(O)(CH₂)₂CH(CO₂CH₂CH₃)NHC(O)CH₃ and —C(O)(CH₂)₂CH(CONH₂)NHC(O)CH₃.

5. A method according to claim 3, wherein R² is selected from —OH, —OCH₃, —OCH₂CH₃, —NH₂, —NHCH₂CO₂H, —NHCH₂CO₂CH₃, —NHCH₂CO₂CH₂CH₃, and —NHCH₂CONH₂.

6. A method according to claim 3, wherein R³ is H or —CH₃.

7. A method according to claim 3, wherein R⁴ is H or —CH₃.

8. A method according to claim 3, wherein the compound of formula (I) is selected from:
N-acetyl cysteine,
N-acetyl cysteine amide,
N-acetyl cysteine ethyl ester,
N-acetyl β,β-dimethyl cysteine ether ester (N-acetylpenicilamine ethyl ester),
N-acetyl β,β-cysteine (N-acetyl penicilamine),
Glutathione ethyl ester,
N-acetyl glutathione ethyl ester,
N-acetyl glutathione,
N-acetyl α-glutamyl ethyl ester cysteinyl glycyl ethyl ester (N-acetyl(β-ethyl ester)glutathione ethyl ester),
N-acetyl α-glutamyl ethyl ester cysteinyl glycine (N-acetyl(β-ethyl ester)glutathione),
γ-glutamyl cysteine ethyl ester,
N-acetyl glutathione amide,
N-acetyl β,β-dimethyl cysteine amide,
N-acetyl β-methyl cysteine amide, and
N-acetyl cysteine glycine amide.

9. A method according to claim 3, wherein the compound of formula (I) is selected from N-acetyl cysteine and N-acetyl cysteine amide.

10. A method of treating a psychiatric or neuropsychiatric disorder according to claim 1, wherein the compound that increases glutathione levels is a glutathione precursor, or a pharmaceutically acceptable salt thereof.

11. A method according to claim 1, wherein the psychiatric or neuropsychiatric disorder is schizophrenia.

12. A method according to claim 1, wherein the psychiatric or neuropsychiatric disorder is major depression.

13. A method according to claim 1, wherein the psychiatric or neuropsychiatric disorder is bipolar disorder.

14. A method according to claim 1, wherein the psychiatric or neuropsychiatric disorder is first episode psychosis.

15. A pharmaceutical composition comprising an antipsychotic drug and a compound that increases glutathione levels, wherein the compound that increases glutathione levels is selected from:
N-acetyl cysteine amide,
N-acetyl cysteine ethyl ester,
N-acetyl β,β-dimethyl cysteine ether ester (N-acetylpenicilamine ethyl ester),
N-acetyl β,β-cysteine (N-acetyl penicilamine),
Glutathione ethyl ester,
N-acetyl glutathione ethyl ester,
N-acetyl glutathione,
N-acetyl α-glutamyl ethyl ester cysteinyl glycyl ethyl ester (N-acetyl(β-ethyl ester)glutathione ethyl ester),
N-acetyl α-glutamyl ethyl ester cysteinyl glycine (N-acetyl(β-ethyl ester)glutathione),
γ-glutamyl cysteine ethyl ester,
N-acetyl glutathione amide,
N-acetyl β,β-dimethyl cysteine amide,
N-acetyl β-methyl cysteine amide, and
N-acetyl cysteine glycine amide.

16-25. (canceled)
